(19) United States
(12) Patent Application Publication
(10) Pub. No.: US 2026/0240662 A1
(43) Pub. Date: Aug. 20, 2026

(54) SYSTEMS AND METHODS FOR EXPANDABLE CORPECTOMY SPACER IMPLANTATION

(71) Applicant: GLOBUS MEDICAL, INC., AUDUBON, PA (US)

(72) Inventors: Dylan McFarland, Pottstown, PA (US); Matthew Bakey, Philadelphia, PA (US); Colm McLaughlin, Glenside, PA (US); David Leff, Philadelphia, PA (US); Samuel Petersheim, Elverson, PA (US); George Howard, Green Lane, PA (US); Derek Changsrivong, San Diego, CA (US); Jody L. Seifert, Birdsboro, PA (US); Anton Gerasimov, San Diego, CA (US); Julia Gambogi, Norristown, PA (US)

(21) Appl. No.: 19/565,820

(22) Filed: Mar. 13, 2026

Related U.S. Application Data
(63) Continuation-in-part of application No. 19/405,875, filed on Dec. 2, 2025.
Application No. 19/405,875 is a continuation of application No. 18/741,898, filed on Jun. 13, 2024, now Pat. No. 12,485,017.
Application No. 18/741,898 is a continuation-in-part of application No. 17/497,001, filed on Oct. 8, 2021, now Pat. No. 12,161,563.
Application No. 17/497,001 is a continuation-in-part of application No. 17/094,177, filed on Nov. 10, 2020, now Pat. No. 11,596,526.
Application No. 17/094,177 is a continuation-in-part of application No. 16/371,419, filed on Apr. 1, 2019, now Pat. No. 10,881,528.
Application No. 16/371,419 is a continuation of application No. 15/836,362, filed on Dec. 8, 2017, now Pat. No. 10,278,834.
Application No. 15/836,362 is a continuation-in-part of application No. 15/264,974, filed on Sep. 14, 2016, now Pat. No. 10,278,833.

(51) Int. Cl. A61F 2/44

(52) U.S. Cl. CPC A61F 2/4465; A61F 2/44; A61F 2/446

(57) ABSTRACT

A corpectomy system includes an insertion instrument for use with an expandable vertebral body replacement implant. The insertion instrument has features which promote robustness and ease of use. The implant assembly includes a core with a threaded actuator, removable endplates configured to engage vertebral bodies, and aa locking mechanism to prevent collapse or movement of the implant assembly after implantation. The insertion instrument operatively engages the foregoing features of the implant assembly. A removal tool may be part of the corpectomy system and used to non-destructively separate the endplates from mechanism automatically engage after removal of an inserter instrument from the implant assembly.

### CROSS-REFERENCE TO RELATED APPLICATION

This application is a continuation-in-part of U.S. patent application Ser. No. 19/405,875, filed Dec. 2, 2025, which is a continuation of U.S. Ser. No. 18/741,898 (now U.S. Pat. No. 12,485,017), filed Jun. 13, 2024, which is a continuation-in-part of U.S. patent application Ser. No. 17/497,001, filed on Oct. 8, 2021 (published as U.S. Pat. Pub. No. 2022-0023066), which is a continuation-in-part of U.S. patent application Ser. No. 17/094,177, filed on Nov. 10, 2020 (now U.S. Pat. No. 11,596,526), which is a continuation-in-part of U.S. Patent Application Ser. No. 16/371,419 (now U.S. Pat. No. 10,881,528), which is a continuation of U.S. patent application Ser. No. 15/836,362, filed on Dec. 8, 2017 (now U.S. Pat. No. 10,278,834), which is a continuation-in-part application of U.S. patent application Ser. No. 15/264,974 filed on Sep. 14, 2016 (now U.S. Pat. No. 10,278,833), the contents of all of which are incorporated by reference herein in their entireties for all purposes.

### FIELD

The present disclosure relates to systems and devices for supporting the spine after removal of at least a part of a vertebra. More particularly, the disclosure relates to vertebral body replacement implant assemblies and attachment assemblies.

### BACKGROUND

Diseases and injury to bone structures, such as the vertebral column, and conditions requiring surgical intervention are relatively common. A variety of conventional implant or graft devices are presently available for use in specific areas. The devices vary in size, shape, materials used, and insertion techniques. For example, in the vertebral column, grafts may provide restoration, decompression, or stabilization of the spine. Typically, these devices include a member that is inserted in the vertebral column to replace an injured portion. An example of such a procedure is a corpectomy, which involves the replacement of all or a portion of the vertebral body with an implant or graft. One exemplary graft is a mesh corpectomy cage which is secured to the adjacent vertebrae via end plates to maintain the position of the implant in situ.

While these conventional devices may generally provide adequate results, they have several disadvantages. For example, often with a corpectomy that involves more than one level, the center segment of the corpectomy cage will settle into a position very close to the patient's dura and spinal cord due to the natural lordosis/kyphosis of the patient. Such proximity to the dura and spinal cord may cause pain, discomfort or further damage to the vertebral column.

Additionally, the endplates are typically secured to the cage with screws. The screws are often cumbersome to install and also make it more difficult to safely remove and replace any component of the construct. Furthermore, there is an inherent risk that the screws may be dropped during a procedure.

With respect to expandable vertebral body replacement (VBR) implants and cages, current expandable VBR cages have limited space for packing bone graft materials and are limited in allowing for bone graft material delivery into the implant after expansion. For example, an expansion range of the shortest expandable cage (15 mm) may only allow for 3-4 mm of expansion.

What is needed is an expandable corpectomy spacer (which may also be used as an interbody spacer) that allows for bone graft material delivery after expansion and allows for up greater expansion for an implant with a starting height of 15 mm, for example.

### SUMMARY

According to one embodiment, an implant assembly for engagement between a first vertebral body and a second vertebral body including an outer ring, a threaded actuator disposed inside the outer ring and having a gear, a right hand end configured to engage the threaded actuator and configured to receive a first endplate, a left hand end configured to engage the threaded actuator and configured to receive a second endplate, and a locking mechanism disposed in the outer ring and configured to removably engage with the gear. The locking mechanism is a single component that is configured to engage with the gear in a locked position and is configured to splay open and disengage with the gear in an unlocked position.

According to one embodiment, a corpectomy system and related methods includes components and features for use in various corpectomy procedures, such as those to replace all or a portion of a vertebral body. In certain implementations, the system includes an implant assembly. The implant assembly includes an outer ring, a threaded actuator disposed inside the outer ring and having a gear, a right hand end configured to engage the threaded actuator and configured to receive a first endplate, a left hand end configured to engage the threaded actuator and configured to receive a second endplate, and a locking mechanism disposed in the outer ring and configured to removably engage with the gear. The locking mechanism is a single component that is configured to engage with the gear in a locked position and is configured to splay open and disengage with the gear in an unlocked position.

The system, in other implementations, may include an insertion instrument configured to move the locking mechanism to the unlocked position and engage the gear to expand or contract the implant assembly.

The insertion instrument likewise is configured to have the implant assembly selectively securable to such insertion instrument and may thus be adapted to surgically implant such implant assembly. In accordance with certain implementations, the insertion instrument includes a split enclosure at its distal end. The split enclosure has a base portion and a housing cover hingedly connected relative to such base portion. The base portion and the housing cover define a housing in which a gearing mechanism is disposed, the gearing mechanism being accessible when the housing cover is open. In still other possible implementations, the housing may include a retaining member which is located within the housing to secure the gearing mechanism from unintended bending moments during operation of the insertion instrument, which bending moments may reduce operative engagement between gears of the gearing mechanism.

In still further implementations, the insertion instrument may include interconnected components to secure the housing cover in a closed position during use of the insertion instrument. Such components, or further components, may likewise be configured to permit such housing cover to be opened upon disconnection of certain components of the insertion instrument.

In certain implementations, for example, the insertion instrument may have inner and outer driving shafts which are selectively removable and thereafter permit the housing cover to be opened. The insertion instrument may further comprise a sensor to sense previous use of the gearing mechanism and an indicator disposed at approximal end of the insertion instrument to indicate such previous use. As such, in response to an indication of previous use, the outer and inner driving shafts may be disconnected and withdrawn from the housing, permitting the housing to be opened, whereupon the gearing mechanism may be accessed, such as for cleaning or other desired purposes.

According to other aspects of the present disclosure, a corpectomy system may include a removal tool for use in associated procedures. In one suitable implementation, the removal tool makes use of a split clamp with two C shaped portions hingedly connected at opposing ends of the portion and thus rotatable relative to each other to open and close the split clamp. When the split clamp is closed, a clamp aperture is defined. The clamp aperture is sized so that, when closed, the removal tool operatively engages in implant assembly received in the aperture, including the core of the implant assembly as well as the tabs of an end cap secured to such implant assembly core.

In one suitable implementation, the removal tool includes a split ring which is connected to the split clamp, the split ring is longitudinally displaceable relative to the split clamp. In order to selectively, longitudinally displace the split ring, the removal tool includes a removal cap which can be threadably advanced to assert forces between the split ring and the split clamp. As such, due to the operative engagement of the split ring and the split clamp with the core and the tabs of an implant assembly received therein, in response to advancement of the removal cap, force may be applied sufficient to induce associated relative movements between the split clamp and the split ring, which movements and associated force disengage, nondestructively, the tabs of the end cap from opposing portions of the core of the implant assembly.

### BRIEF DESCRIPTION OF THE DRAWINGS

The accompanying drawings, which are incorporated herein and constitute part of this specification, illustrate the presently preferred embodiments of the disclosure, and, together with the general description given above and the detailed description given below, serve to explain the features of the disclosure. In the drawings:

FIG. 1 is a side elevation view of an implant assembly according to an exemplary embodiment attached between vertebrae.

FIG. 2 is a perspective view of an exemplary cage member of the implant assembly of FIG. 1.

FIG. 3 is a top plan view of the cage member of FIG. 2.

FIG. 4 is a top plan view of an alternative cage member.

FIG. 5 is a top plan view of yet another alternative cage member.

FIG. 6 is an exploded perspective view of the implant assembly of FIG. 1.

FIG. 7 is an exploded perspective view of another exemplary implant assembly.

FIG. 8 is a perspective view of the implant assembly of FIG. 7 in an assembled configuration.

FIG. 9 is a cross-sectional view along the line 9-9 in FIG. 8.

FIG. 10 is a perspective view of an exemplary cage member.

FIGS. 11A-11C are perspective views of exemplary endcaps.

FIGS. 12A-12C are perspective views of exemplary endcaps.

FIG. 13A is a top view of an exemplary endcap.

FIG. 13B is a perspective view of an exemplary endcap

FIG. 14A is perspective view of an exemplary implant assembly prior to installation of exemplary endcaps.

FIG. 14B is a perspective view of an exemplary implant assembly.

FIGS. 15A-15B are perspective views of an exemplary removal tool.

FIG. 16 is a perspective view of an exemplary inner core of an expandable vertebral body replacement implant.

FIGS. 17A and 17B are perspective views of an expandable vertebral body replacement implant.

FIG. 18 is a perspective views of an expandable vertebral body replacement implant in an expanded configuration.

FIG. 19A is a top view of an expandable vertebral body replacement implant.

FIG. 19B is a perspective view of an expandable vertebral body replacement implant.

FIG. 19C FIG. 19A is a top view of an expandable vertebral body replacement implant with an attached removable end plate.

FIGS. 20A-20B are perspective views of an exemplary removable end plate for an expandable vertebral body replacement implant.

FIG. 21 is a perspective view of a removable endplate detached from an expandable vertebral body replacement implant.

FIGS. 22A-22B are perspective view of a removable endplate attached to an expandable vertebral body replacement implant.

FIGS. 23A-B are perspective views of an expandable vertebral replacement implant.

FIG. 24 is an exploded view of an expandable vertebral replacement implant.

FIGS. 25A-D are perspective views of a locking mechanism for an expandable vertebral body replacement implant.

FIGS. 26A-B are top views of expandable vertebral replacement implant.

FIGS. 27A-B show perspective views of an expandable vertebral replacement implant.

FIG. 28 is an exploded view of an expandable vertebral replacement implant.

FIG. 29 shows a perspective view of an expandable vertebral replacement implant with an insertion instrument.

FIG. 30A shows a threaded actuator and a locking mechanism for an expandable vertebral replacement implant.

FIG. 30B shows an insertion instrument engaged with an expandable vertebral replacement implant.

FIG. 31 shows a top view of a threaded actuator and a locking mechanism of an expandable vertebral replacement implant.

FIG. 32A shows a threaded actuator and a locking mechanism for an expandable vertebral replacement implant.

FIG. 32B shows an insertion instrument engaged with an expandable vertebral replacement implant.

FIG. 33 shows an expandable vertebral replacement implant.

FIGS. 34A-B show perspective views of an expandable vertebral replacement implant.

FIG. 35 is an exploded view of an expandable vertebral replacement implant.

FIG. 36 shows an insertion instrument engaged with an expandable vertebral replacement implant.

FIG. 37A shows a threaded actuator and a locking mechanism for an expandable vertebral replacement implant.

FIG. 37B shows an insertion instrument engaged with an expandable vertebral replacement implant.

FIG. 38 shows a top view of a threaded actuator and a locking mechanism of an expandable vertebral replacement implant.

FIG. 39 shows a threaded actuator and a locking mechanism for an expandable vertebral replacement implant.

FIG. 40 is a side, elevational view of another potential implementation of an insertion instrument of a corpectomy system according to still other aspects of the present disclosure.

FIG. 41 is an enlarged, perspective view of a distal portion of the insertion instrument of FIG. 40.

FIG. 42 is a perspective view of the distal end of the insertion instrument of FIGS. 40 and 41 from another orientation showing the housing cover opened, according to certain implementations of the present disclosure.

FIG. 43 is another perspective view of the distal end of the insertion instrument shown in FIGS. 40-42.

FIG. 44 is an exploded, perspective view of the distal end of the insertion instrument shown in FIGS. 40-43.

FIG. 45 is a side, sectional view of the distal end of the instrument shown in FIGS. 40-44.

FIG. 46 is a side elevational view of the proximal end, according to one possible implementation of the instrument of FIGS. 40-45.

FIG. 47 is an exploded, perspective view of one possible implementation of a removal tool which may be part of various implementations of a corpectomy system according to the present disclosure.

FIG. 48 is a perspective view of the removal tool shown in FIG. 47.

FIG. 49 is a rear elevational view of the removal tool shown in FIGS. 47, 48.

FIG. 50 is a cross-sectional, elevational view of another possible implementation of the removal tool according to the present disclosure, shown attached to an exemplary implant assembly.

FIG. 51 is a front, elevational view of the removal tool of FIGS. 47-50, according to still another potential implementation.

FIG. 52 is an enlarged, sectional view of the removal tool of FIGS. 47-51 in operative engagement with an end cap and core according to one possible implementation of the present disclosure.

FIG. 53 is a side, elevational view of another possible implementation of the removal tool according to the present disclosure.

FIG. 54 is a front, elevational view of the removal tool shown in FIG. 53.

### DETAILED DESCRIPTION

In the drawings, like numerals indicate like elements throughout, with alphabetical or prime identifiers indicating a particular one of the more generally identified element. Certain terminology is used herein for convenience only and is not to be taken as a limitation on the present invention. The following describes preferred embodiments of the present disclosure. However, it should be understood, based on this disclosure, that the invention is not limited by the preferred embodiments described herein.

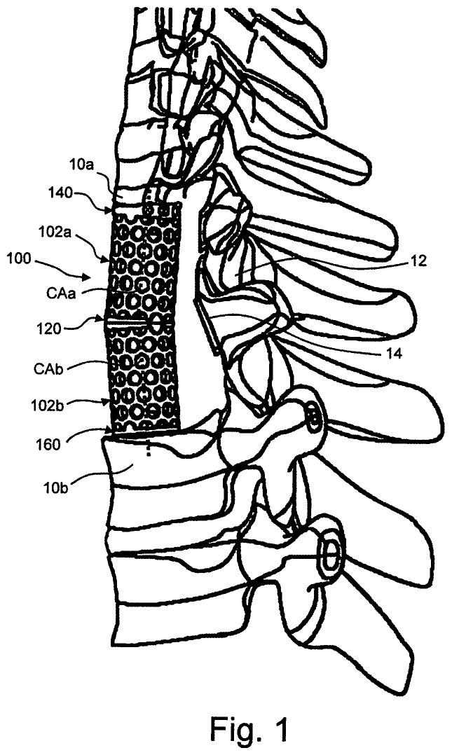

Referring to FIG. 1, an exemplary implant assembly 100 in accordance with an embodiment of the disclosure is illustrated positioned between a pair of vertebrae 10a, 10b. The implant assembly 100 generally includes two or more mesh cages 102a, 102b, at least one intermediate plate 120 and a pair of endplates 140, 160. The intermediate plate 120 is secured between the cages 102a, 102b and has an angled configuration such that the central axis CAa of the cage 102a is angled at an acute angle α (see FIG. 9) relative to the central axis Cab of the cage 102b. The endplate 140 is secured to the end of cage 102a and secures the cage 102a to the adjacent vertebrae 10a. The endplate 160 is secured to the end of cage 102b and secures the cage 102b to the adjacent vertebrae 10b. The lordotic angle α created by the intermediate plate 120 helps to align the ends of the cages 102a, 102b with the vertebral endplates 10a, 10b which will help in minimizing subsidence. The lordotic angle α also positions the cages 102a, 102b away from the dura 14 and spinal cord 12 of the patient.

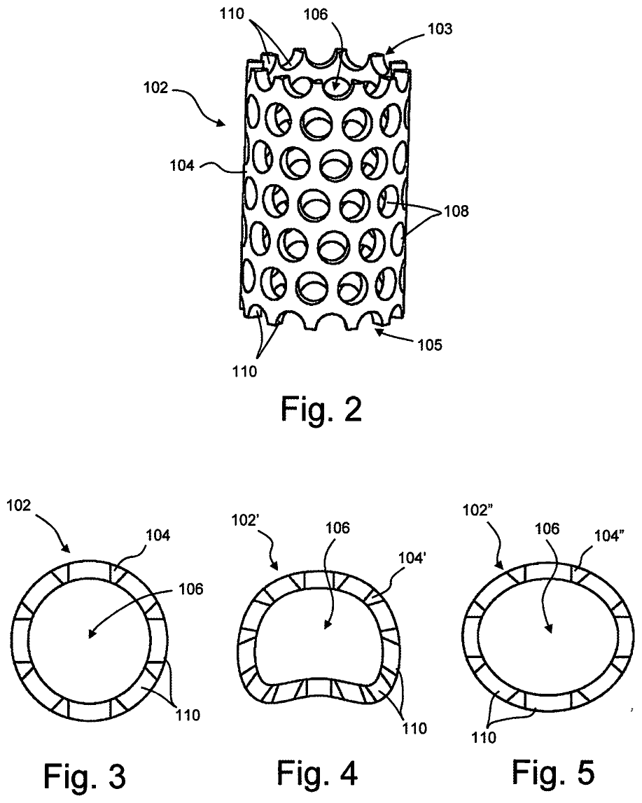

Referring to FIGS. 2-5, exemplary embodiments of the cage 102 will be described. Each cage 102 generally has a hollow tubular body 104 extending between ends 103, 105 with a passage 106 therethrough. The tubular body 104 may be manufactured from various materials, for example, but not limited to, titanium or other metals, carbon fibers, ceramics, polymers or biocomposites. As illustrated in the embodiment of FIGS. 2 and 3, the exemplary cage 102 has a circular cross-section, however, the cage 102 may have various configurations. As two non-limiting examples, the cage 102′ illustrated in FIG. 4 has a kidney shaped cross-section and the cage 102″ illustrated in FIG. 5 has an oval cross-section. The mesh cages 102 may be supplied at various convenient lengths or can be cut to size. It is understood that the cages 102a, 102b of the implant assembly 100 may have the same or different lengths.

The tubular body 104 defines a series of radial openings 108 which open into the through passage 106. The radial openings 108 facilitate bone ingrowth and provide connection points for clips on the intermediate plate 120 and the endplates 140, 160, as will be described in more detail hereinafter. The openings 108 are preferably evenly spaced about the tubular body 104 to create a mesh thickness that offers compressive and torsional strength while allowing the cage to be easily cut to length. While the openings 108 are illustrated with a circular configuration, they openings 108 may have other shapes, for example, square or octagon.

Each end 103, 105 of the cage 102 includes a series of end openings 110 which open into the through passage 106 and are also open to the respective end surface of the tubular body 104. The end openings 110 are configured to receive tabs extending from the intermediate plate 120 and the endplates 140, 160 to provide rotational stability. While the end openings 110 are illustrated with a semi-circular configuration, they openings 108 may have other shapes, for example, square or octagon, and may have a depth that is more or less than one-half the width.

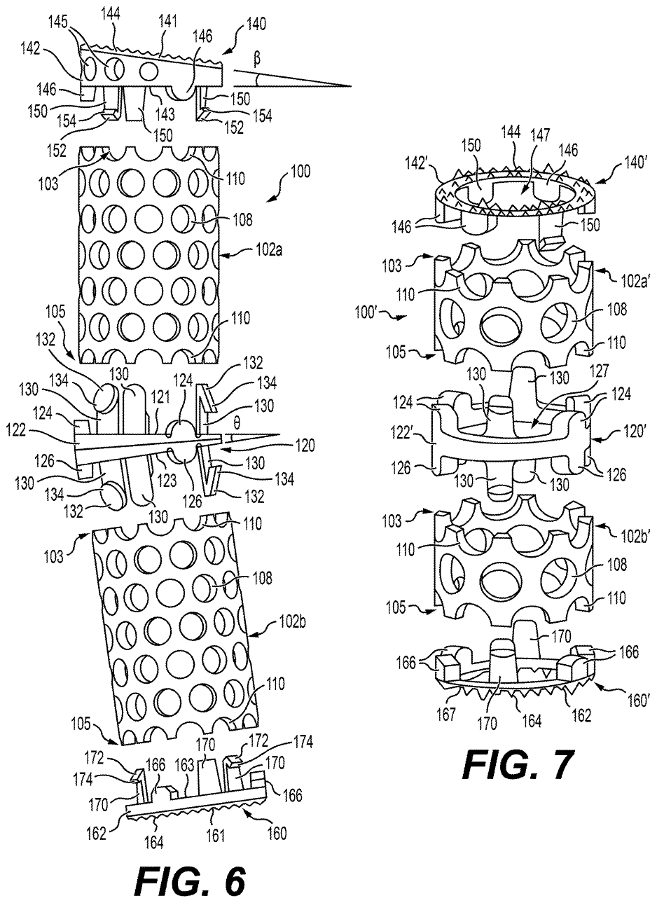

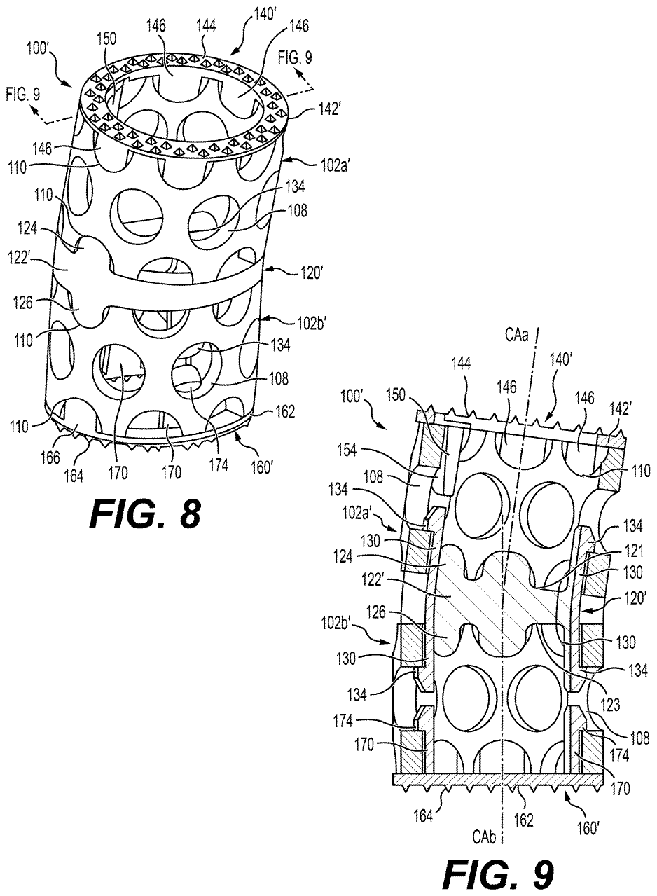

Exemplary intermediate plates 120, 120′ and endplates 140, 140′, 160, 160′ will be described with reference to FIGS. 6-9. The differences between the components of the implant assembly 100 of FIG. 6 and the implant assembly 100′ of FIGS. 7-9 will be identified, otherwise the components are substantially the same. With respect to the cages, the cages 102a′ and 102b′ are shorter than the cages 102a and 102b and have an oval configuration instead of the round configuration of the cages 102a and 102b.

Turning to the intermediate plates 120, 120′, each plate 120, 120′ has a ring shaped body 122, 122′ with a passage 127 therethrough. The body 122 has a circular configuration to match that of the cages 102a, 102b while the body 122′ has an oval configuration to match that of the cages 102a′, 102b′. Each body 122, 122′ extends between opposed contact surfaces 121, 123. The contact surfaces 121, 123 are at an angle θ relative to one another. This angle θ between the contact surfaces 121, 123 creates the lordotic angle α between the central axes CAa and Cab of the cages. In the event that more than two cages are utilized, intermediate plates 120 can be positioned between respective cages 102, each with the same or different angles θ.

On each body 122, 122′, a plurality of tabs 124 extend from the contact surface 121 and a plurality of tabs 126 extend from the contact surface 123. The tabs 124, 126 have shapes which complement the shape of the end openings 110 such that the tabs 124, 126 are received in and engage the end openings 110 of the respective cages 102. Engagement between the tabs 124, 126 and the end openings 110 provides rotational stability between the intermediate plates 120, 120′ and the cages 102. As seen in comparing the intermediate plate 120 with the intermediate plate 120′, the number and location of tabs 124, 126 may be varied. Additionally, the tabs 124, 126 may be eliminated provided the spring clips 130, described below, provide sufficient rotational stability.

A plurality of spring clips 130 extend from each contact surface 121, 123. As seen in comparing the intermediate plate 120 with the intermediate plate 120′, the number and location of spring clips 130 may be varied. Each spring clip 130 includes a body 132 extending from the respective surface 121, 123 and defining a retaining ledge 134 spaced from the respective surface 121, 123. The bodies 132 may have different lengths to account for the angle between the contact surfaces 121, 123 such that each of the retaining ledges 134 on respective side of the intermediate plate 120, 120′ are co-planar. With the retaining ledges 134 co-planar, the retaining ledges 134 will engage a common row of openings 108 in a respective cage 102 (see FIG. 9). Each spring clip body 132 is elastic such that it bends inward as spring clips 130 pass into the cage through passage 106, but then springs outward as the retaining ledge 134 aligns with a respective opening 108. The bodies 132 may have a tapered end surface to promote the inward bending of the spring clips 130 as they are inserted. The retaining ledges 134 thereby engage the openings 108 and axially secure the intermediate plate 120, 120′ to the cages 102. If it is desired to remove the intermediate plate 120, 120′ from the cages 102, the retaining ledges 134 are biased inward until they clear the openings 108 and the intermediate plate 120, 120′ is easily disconnected.

Turning to the endplates 140, 140', each plate 140, 140′ has a ring shaped body 142, 142′ with a passage 147 therethrough. The body 142 has a circular configuration to match that of the cage 102a while the body 142′ has an oval configuration to match that of the cage 102a′. Each body 142, 142′ extends between opposed contact surfaces 141, 143, with the contact surface 141 being a bone contact surface and the contact surface 143 being a cage contact surface. The contact surfaces 141, 143 of the endplate 140 are at an angle β relative to one another while the contact surfaces 141, 143 of the endplate 140′ are parallel to one another. The endplates 140, 140′ can have an angled or parallel configuration. This angle θ, or lack of angle, between the contact surfaces 141, 143 allows the surgeon to make an implant assembly 100, 100′ unique to the patient's anatomy.

On each body 142, 142′, a plurality of projections 144 or the like extend from the contact surface 141 and are configured to engage the vertebrae contact surface. Various surface configurations may be utilized to achieve a desired securement with the vertebrae contact surface. Additionally, the body 142 may include radial openings 145 which promote bone growth into the endplate 140.

Similar to the intermediate plates, a plurality of tabs 146 extend from the contact surface 143. The tabs 146 have shapes which complement the shape of the end openings 110 such that the tabs 146 are received in and engage the end openings 110 of the respective cages 102. Engagement between the tabs 146 and the end openings 110 provides rotational stability between the endplates 140, 140′ and the cages 102. As seen in comparing the endplate 140 with the endplate 140′, the number and location of tabs 146 may be varied. Additionally, the tabs 146 may be eliminated provided the spring clips 150, described below, provide sufficient rotational stability.

A plurality of spring clips 150 extend from the contact surface 143. As seen in comparing the endplate 140 with the endplate 140', the number and location of spring clips 150 may be varied. Each spring clip 150 includes a body 152 extending from the surface 143 and defining a retaining ledge 154 spaced from the surface 143. With the endplate 140, the bodies 152 may have different lengths to account for the angle between the contact surfaces 141, 143 such that each of the retaining ledges 154 of the intermediate plate 140 are co-planar. With the endplate 140', the bodies 152 will have a common length such that the retaining ledges 154 are co-planar. With the retaining ledges 154 co-planar, the retaining ledges 154 will engage a common row of openings 108 in a respective cage 102 (see FIG. 9). Each spring clip body 152 is elastic such that it bends inward as spring clips 150 pass into the cage through passage 106, but then springs outward as the retaining ledge 154 aligns with a respective opening 108. The bodies 152 may have a tapered end surface to promote the inward bending of the spring clips 150 as they are inserted. The retaining ledges 154 thereby engage the openings 108 and axially secure the endplate 140, 140′ to the cage 102. If it is desired to remove the endplate 140, 140′ from the cage 102, the retaining ledges 154 are biased inward until they clear the openings 108 and the endplate 140, 140′ is easily disconnected.

Turning to the endplates 160, 160′, each plate 160, 160′ has a ring shaped body 162, 162′ with a passage 167 therethrough. The body 162 has a circular configuration to match that of the cage 102a while the body 162′ has an oval configuration to match that of the cage 102a′. Each body 162, 162′ extends between opposed contact surfaces 161, 163, with the contact surface 161 being a bone contact surface and the contact surface 163 being a cage contact surface. In the illustrated embodiments, the contact surfaces 161, 163 of each of the endplates 160, 160′ are parallel to one another, however, it is understood that the surfaces 161, 163 may be angled relative to one another to allow the surgeon to make an implant assembly 100, 100′ unique to the patient's anatomy.

On each body 162, 162′, a plurality of projections 164 or the like extend from the contact surface 161 and are configured to engage the vertebrae contact surface. Various surface configurations may be utilized to achieve a desired securement with the vertebrae contact surface.

Similar to the intermediate plates, a plurality of tabs 166 extend from the contact surface 163. The tabs 166 have shapes which complement the shape of the end openings 110 such that the tabs 166 are received in and engage the end openings 110 of the respective cages 102. Engagement between the tabs 166 and the end openings 110 provides rotational stability between the endplates 160, 160′ and the cages 102. As seen in comparing the endplate 160 with the endplate 160′, the number and location of tabs 166 may be varied. Additionally, the tabs 166 may be eliminated provided the spring clips 170, described below, provide sufficient rotational stability.

A plurality of spring clips 170 extend from the contact surface 163. As seen in comparing the endplate 160 with the endplate 160′, the number and location of spring clips 170 may be varied. Each spring clip 170 includes a body 172 extending from the surface 163 and defining a retaining ledge 174 spaced from the surface 163. With each of the endplates 160, 160′, the bodies 172 will have a common length such that the retaining ledges 174 are co-planar. With the retaining ledges 174 co-planar, the retaining ledges 174 will engage a common row of openings 108 in a respective cage 102 (see FIG. 9). Each spring clip body 172 is elastic such that it bends inward as spring clips 170 pass into the cage through passage 106, but then springs outward as the retaining ledge 174 aligns with a respective opening 108. The bodies 172 may have a tapered end surface to promote the inward bending of the spring clips 170 as they are inserted. The retaining ledges 174 thereby engage the openings 108 and axially secure the endplate 160, 160′ to the cage 102. If it is desired to remove the endplate 160, 160′ from the cage 102, the retaining ledges 174 are biased inward until they clear the openings 108 and the endplate 160, 160′ is easily disconnected.

Upon assembly of the implant assemblies 100, 100′, as illustrated in FIGS. 1 and 8-9, the integrated clips 130, 150, 170 on the intermediate plate and endplates snap into the corresponding holes 108 in the mesh cages 102 for a secure fit. The quick clip system makes a secure construct while allowing for components to be removed and replaced prior to insertion into the body should the need arise. The intermediate plate 120 offers a safe and secure connection to the mesh cages 102 while providing lordosis/kyphosis at the center of the construct instead of at the end of the cage only. This allows for the body of the implant assembly to be moved away from the dura and spinal cord of the patient.

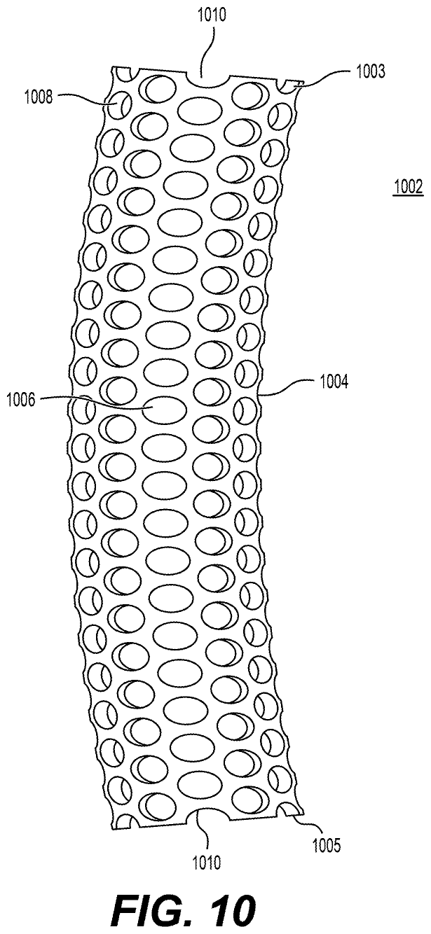

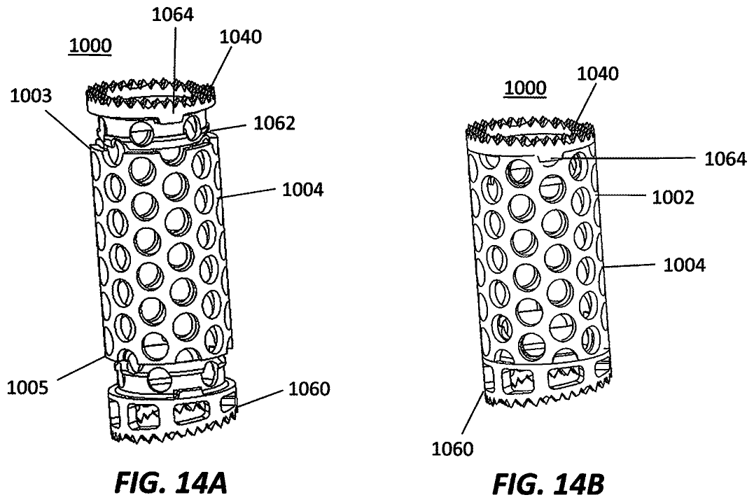

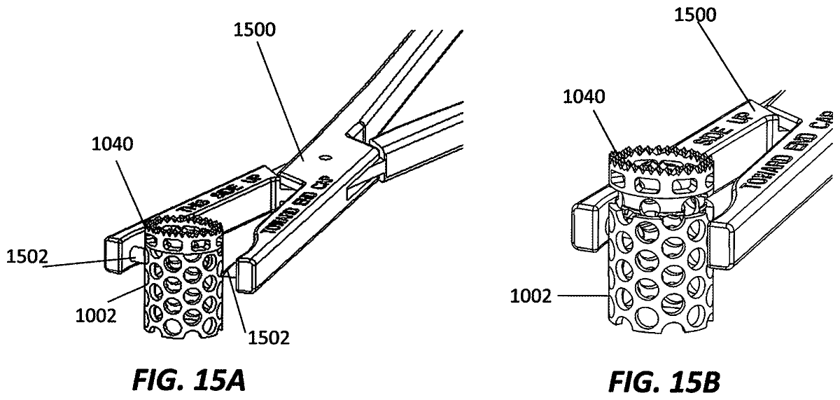

In a further embodiment, FIGS. 10-15B relate to components for an implant assembly 1000 (shown in FIGS. 15A-15B). Implant assembly 1000 is similar in structure to implant assembly 100 but does not contain, at least, the intermediate plates as discussed above. In FIGS. 10-15B, a cage 1002 generally has a hollow tubular body 1004 extending between ends 1003, 1005 with a passage 1006 therethrough. The tubular body 1004 may be manufactured from various materials, for example, but not limited to, titanium or other metals, carbon fibers, ceramics, polymers or biocomposites. Similar to the cage 102, cage 1002 may have a circular cross-section, a kidney shaped cross-section, or an oval cross-section as shown in FIGS. 2-5. The cage 1002 may be supplied at various convenient lengths or can be cut to size. Cage 1002 is illustrated as being curved so that a center section of cage 1002 may be positioned away from dura and spinal cord as previously discussed with respect to cage 102 shown in FIG. 1.

The tubular body 1004 may define a series of radial openings 1008 which open into the passage 1006. The radial openings 1008 may facilitate bone ingrowth and provide connection points for endplates 1040, 1060, as will be described in more detail hereinafter. The openings 1008 are preferably evenly spaced about the tubular body 1004 to create a mesh thickness that offers compressive and torsional strength while allowing the cage to be easily cut to length. While the openings 1008 are illustrated with a circular configuration, the openings 1080 may have other shapes, for example, square or octagon.

Each end 1003, 1005 of the cage 1002 includes at least one end opening 1010 which opens into the through passage 1006 and is also open to the respective end surface of the tubular body 1004. The end openings 1010 are configured to receive a tab 1064 extending from the endplates 1040, 1060 to provide rotational stability. While the end openings 1010 are illustrated with a semi-circular configuration, the openings 1010 may have other shapes, for example, square or octagon, and may have a depth that is more or less than one-half the width. Endplates 1040 and 1060 may be configured to have a tapered end 1062 that allows a tapered lead in point for the endplate 1040, 1060 when inserted into cage 1002. Endplates 1040 and 1060 are configured to press-fit or snap-fit into an end of cage 1002. A final fit may be achieved when an underside of an endplate is flush with an end of cage 1002 and tab 1064, which may be an anti-torsion tab, is seated in a partial hole, such as opening 1010.

The end caps may be angled with optional heights that a surgeon may use to make an implant assembly (e.g., implant assembly 1000) that is tailored to a specific patient's anatomy. Endcaps 1040 and 1060 may be accurately centered to each other and the cage 1002 by means of radial holes 1008 in cage 1002.

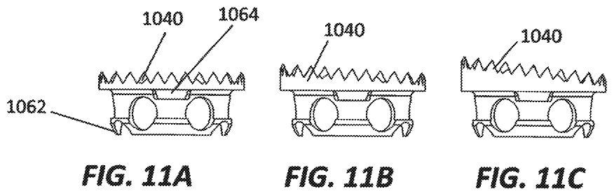

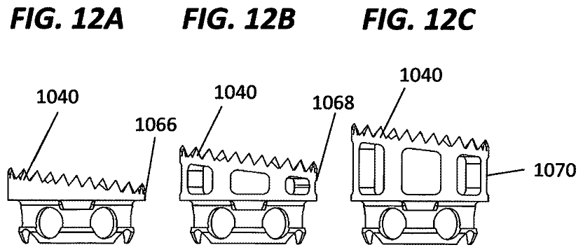

As shown in FIGS. 11A-12C, endcaps 1040 (and endcaps 1060) may have a variety of lordotic options that will allow a surgeon to choose an end cap that will closely match the lordosis of the patient. FIGS. 11A-C illustrate varying angles of endcap 1040. FIGS. 12A-C illustrate varying heights of endcap 1040. Further, each end cap 1040, 1060 may be available in a number of height options. For example, FIG. 12A may correspond to a height 1066 of 1.5 mm, FIG. 12B may correspond to a height 1068 of 3.5 mm, and FIG. 12C may correspond to a height 1068 of 5.5 mm. Varying height options may allow a surgeon to quickly remove and replace an end cap 1040 in the case when an implant assembly 1000 is too short or too long. An end cap removal tool 1500 may be supplied to facilitate the change. For example, as shown in FIGS. 15A-15B, removal tool 1500 may include opposing projections 1502 that are configured in a way to enter one of openings 1008 to engage tapered ends 1064 of the applicable endplate to remove the endplate out of cage 1002.

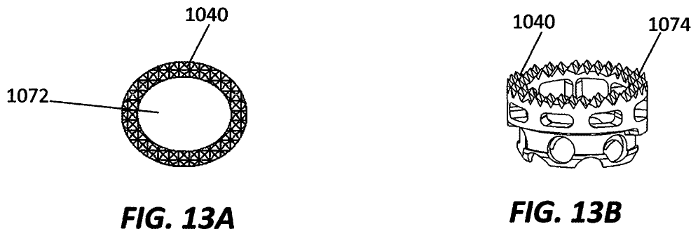

As shown in FIGS. 13A-13B, an inner geometry 1072 of end cap 1040 may be free of mechanical protrusions, which may allow for a maximum graft window for insertion of bone growth material. A top surface of end cap 1040 may contain teeth 1074 (with or without a laser etched surface) that may aid implant assembly 1000 to grip a vertebral endplate and promote bony ingrowth.

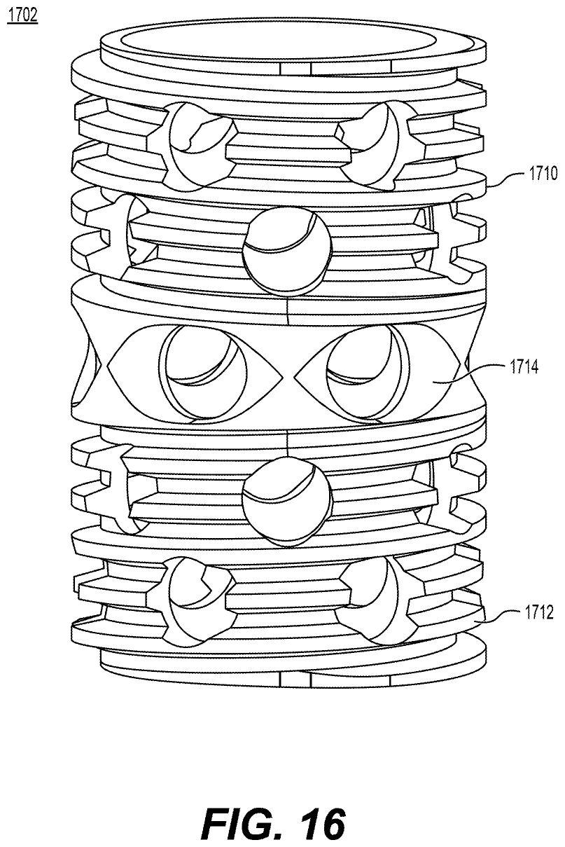

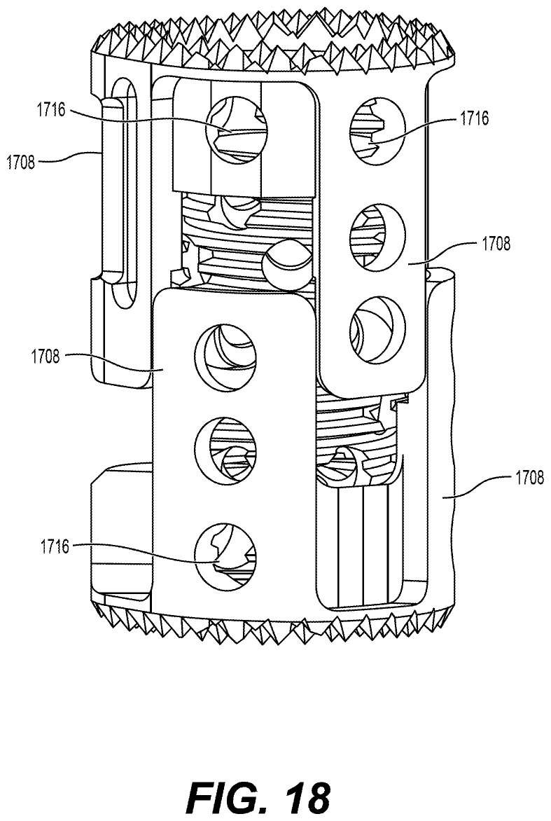

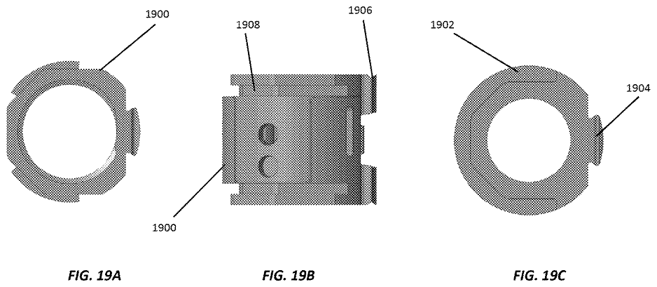

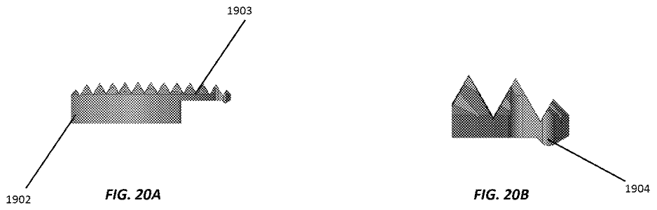

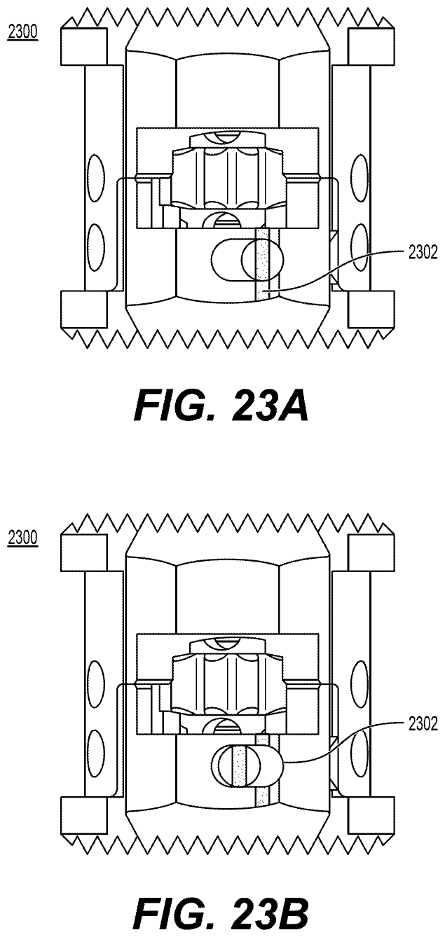

Referring now to FIGS. 16-18, an exemplary implant assembly 1700 in accordance with an embodiment of the disclosure is illustrated. Assembly 1700 is an expandable vertebral body replacement (VBR) implant. It may be used for corpectomies or as another type of interbody implant. Assembly 1700 may include three components. One component may be an inner core 1702 with a left-hand thread at one end and a right-hand thread at the opposite end. The other two components may be two outer cores 1704, 1706, one with left-hand internal threads and one with right-hand internal threads. Each outer core 1704, 1706 has mating rails 1708 (or fingers) that prevent torsional spin while allowing expansion, as illustrated in FIG. 18. Assembly 1700 may be locked into final position by a variety of mechanisms, including as described in greater detail with respect to FIGS. 23A-26.

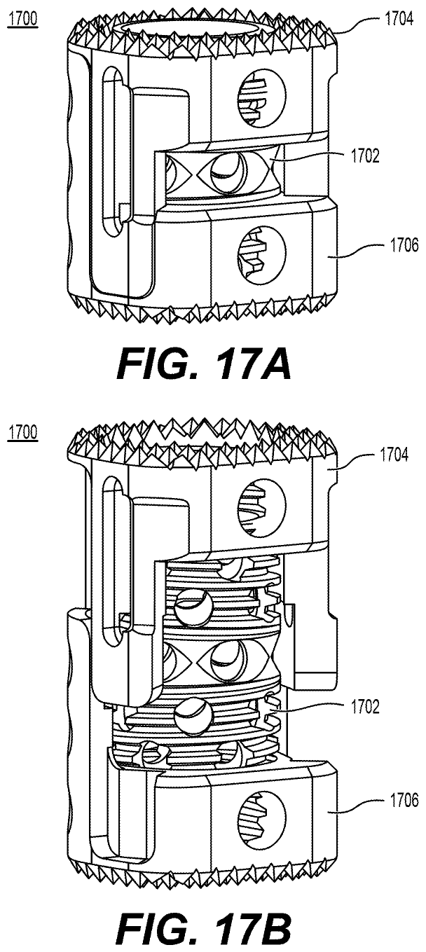

An exemplary inner core 1702 is illustrated in FIG. 16. Inner core 1702 may have a left-handed thread at one end 1710 and a right-handed thread at the opposite end 1712. As inner core 1702 is turned in one direction by an inserter instrument via the set of center holes 1714, the implant 1700 expands as illustrated in FIGS. 17A and 17B. As an example, implant 1700 may allow for up to 8 mm of expansion for an implant with a starting height of 15 mm. Turned in the opposite direction, the implant contracts. The rails 1708 on outer cores 1704, 1706 mate to allow the outer cores 1704, 1706 to expand and prevent spin. Implant 1700 may be configured to be packed with bone graft material after expansion which may aid in fusion of the vertebral bodies through windows 1716.

Referring now to FIGS. 19A-22B, illustrated are exemplary embodiments of removable endplates 1902 for an expandable VBR assembly. By way of background, current VBR cages have endplates that attach to the end of the cage with screws. These screws may be easily dropped and add an extra step to expandable VBR assembly 1700 or 1900.

FIG. 19A illustrates an exemplary expandable VBR assembly 1900 from a top view and FIG. 19B illustrates assembly 1900 in a collapsed configuration from the side. FIG. 19C shows an underside of endplate 1902. Endplate 1902 may be an endplate that slides onto the end of the implant 1900 and configured to snap into place. The may be achieved by using a machined geometric relief 1904 on endplate 1902 and counter-relief 1906 on the implant assembly 1900. Endplate 1902 may slide into place with the use of a standard dado or undercut 1908. Undercut 1908 may be configured to prevent endplate 1902 from lifting off of assembly 1900.

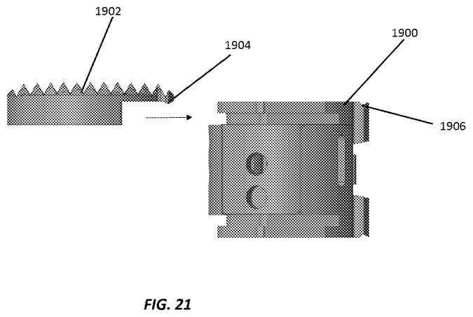

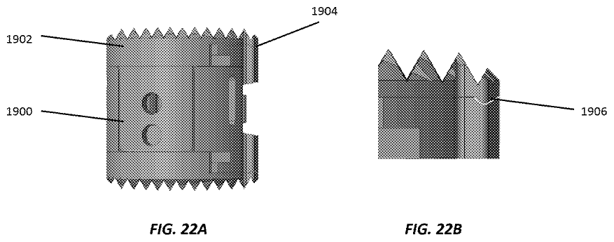

FIG. 20A illustrates a side view of endplate 1902 and FIG. 20B illustrates an expanded view of relief 1904 of endplate 1902. Endplate 1902 may have protrusions 1903 that may engage a vertebral body. As shown in FIG. 21, endplate 1902 is configured to attach to implant assembly 1900 by sliding on either end of assembly 1900. When fully attached, endplate 1902 snaps on the assembly 1900 as illustrated in FIG. 22A. FIG. 22B illustrates an expanded view of relief 1904 and counter-relief 1906 after attachment of endplate 1902. One advantage of the snap is that it eliminates the need for a separate screw to be used to hold endplate 1902 in place on the assembly 1900. It may also eliminates the need of an assembly block or fixture.

Referring now to FIGS. 23A-26B, an exemplary expandable VBR assembly 2300 consistent with the principles of the present disclosure is illustrated. Here, assembly 2300 includes a locking mechanism 2302. By way of background, expandable VBR cages need to be locked so as to prevent them from collapsing or losing height due to the natural vibration and/or movement of the human body into which they are implanted. Current implants available often use a locking screw that needs to be manually locked by the surgeon as a final step of the surgery. Due to the location in the body and approach of a corpectomy and the small size of the implant, the small size of the locking screw head and accessibility to it may prove to be difficult for the surgeon to locate, align and lock.

As shown in FIGS. 23A-26B, assembly 2300 may have locking mechanism 2302, which may be an automatic locking system for a surgical implant that securely locks the implant when the instrument that is used to insert the implant into the body is removed. This may eliminate the need to manually lock the implant as a final surgical step.

Assembly 2300 may have locking mechanism 2302 to automatically lock assembly 2300 in any position to prevent it from moving, winding down or collapsing from weight, external interference, movement or vibration. Locking mechanism may be seated within the interior portion of assembly 2300, with a visible gage to indicate that locking mechanism 2302 is locked or unlocked. The lock functions in conjunction with right-hand and left-hand threads as described earlier. The implant may contain one or two locks depending on size. For purpose of illustration, one lock is shown.

As shown in FIGS. 23A and 23B, when locking mechanism is engaged, an etched or engraved lines will be aligned to visually form a solid line as shown in FIG. 23A. In the unlocked position, the lines will appear separated or perpendicular to one another, as shown in FIG. 23B.

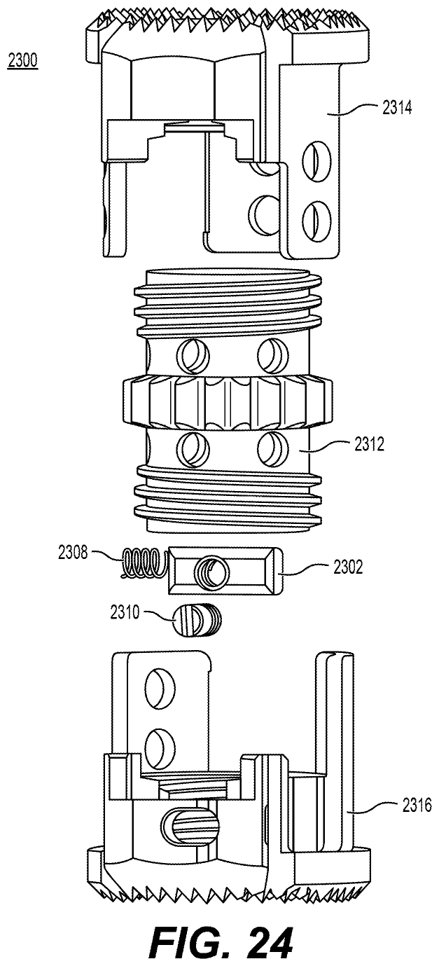

An exploded view of assembly 2300 is illustrated in FIG. 24. Assembly 2300 may include locking mechanism 2302, a spring mechanism 2308, a lock indicator 2310, inner core 2312 which may function as a threaded actuator as previously described, an outer core 2314 (right handed (RH) end), and an outer core 2316 (left handed (LH) end).

In practice, locking mechanism 2302 may be positioned inside of a cavity in outer core 2314 (RH end) or outer core 2316 (LH End) of assembly 2300. Spring mechanism 2308 assists by keeping constant tension on locking mechanism 2302 in the “locked” position. Spring mechanism 2308 is illustrated as a coiled compression spring. It may also take the form of a constant tension spring, a leaf spring, or any other form that exerts pressure against the locking mechanism 2302. Locking mechanism 2302 is disengaged into the un-locked position by a portion of the insertion instrument in the form of a wedge, clamp or screw, thereby allowing the implant to be expanded or contract by means of a drive gear. When the insertion instrument is removed from assembly 2300, locking mechanism 2302 is engaged as spring mechanism 2308 pushes locking mechanism 2302 into the locked position.

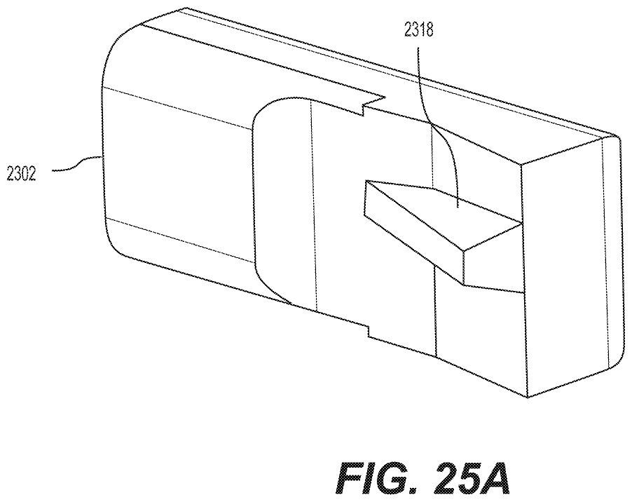

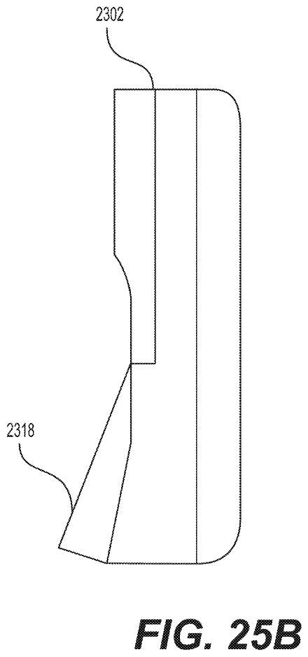

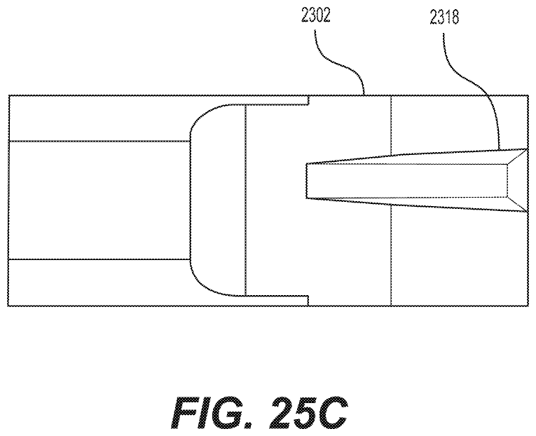

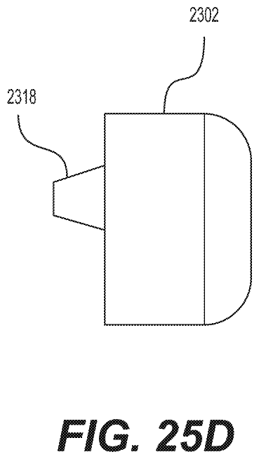

As illustrated in FIGS. 25A-25D, locking mechanism 2302 may be a tapered block tangent to the major diameter of the inner core 2312, with a centered tooth 2318, or multiple teeth, that are oversized to the minor diameter of inner core 2312. As locking mechanism 2302 is pushed into position, it wedges itself against the inner core 2312 and outer core 2314 (RH end) or outer core 2316 (LH end) to act as a “doorstop,” preventing assembly 2300 from further movement.

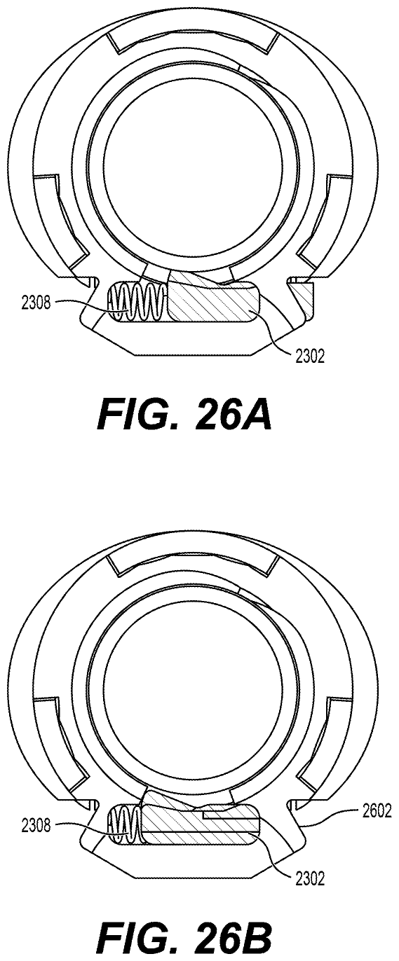

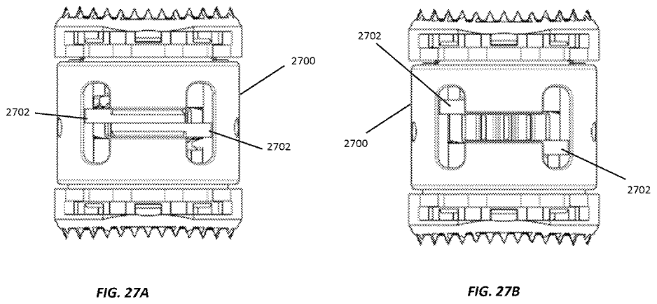

FIG. 26A illustrates assembly 2300 in a locked position and FIG. 26B shows assembly 2300 in an unlocked position. At instrument insertion point 2602, an instrument pushes against locking mechanism 2302 to unlock assembly 2300 to expand or contract assembly 2300. One advantage of this configuration is that the locking of assembly 2300 occurs automatically once the insertion instrument is removed. The lock is not subject to surgical approach, visual alignment or physical access to a separate locking screw that may be difficult for the surgeon to see.

Referring now to FIGS. 27A and 27B, illustrated is an assembly 2700 consistent with the principles of the present disclosure. Assembly 2700 may contain the same or similar components as previously noted and contains a locking mechanism 2702 to automatically lock assembly 2700. Locking mechanism 2702 may automatically lock a threaded implant in any position to prevent it from moving, winding down or collapsing from weight, external interference, movement or vibration. Locking mechanism 2702 is seated within an interior portion of assembly 2700, with a visible gage to indicate that locking mechanism 2702 is in a locked or unlocked position. The lock functions in conjunction with the drive gear of the threaded actuator. Assembly 2700 may contain one or two locks depending on size.

When the automatic locking mechanism 2702 is engaged, locking mechanism visually covers the gear teeth of the actuator. In the unlocked position, the lock is pushed off of the gear into a neutral zone, allowing the gear of the inserter to mate to the gear of the implant, allowing the actuator to turn and the implant to expand and contract. In FIG. 27A, assembly 2700 is shown in a locked position with locking mechanism 2702 hiding the gear teeth of assembly 2700. FIG. 27A shows assembly 2700 in an unlocked position and wherein the gear teeth of assembly 2700 are visible to a user.

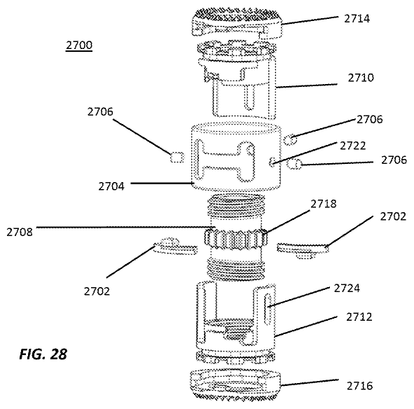

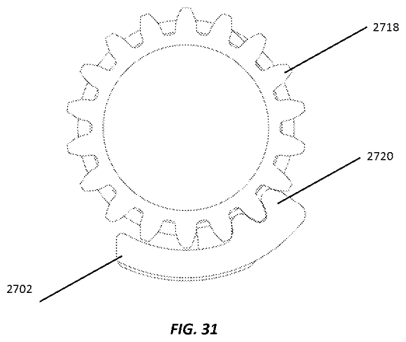

An exploded view of assembly 2700 is shown in FIG. 28. Assembly 2700 may include locking mechanism 2702, an outer ring 2704, one more retaining pins 2706, a threaded actuator 2708, a right hand (RH) end 2710, a left hand (LH) end 2712, and endplates 2714, 2716 configured to engage vertebral bodies. Threaded actuator 2708 may have gear 2718. Locking mechanism may have teeth 2720 as shown in FIG. 31. Outer ring 2704 may have a mating hole 2722 configured to receive one of retaining pins 2706. Each of RH end 2710 and LH end 2712 may have a mating slot 2724.

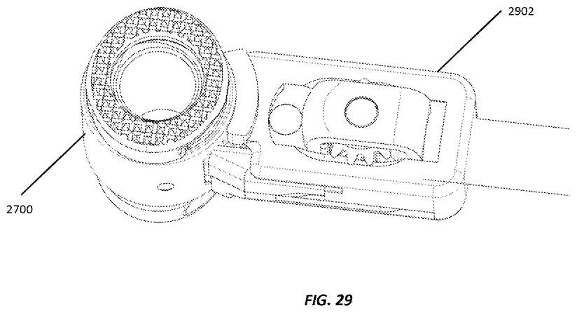

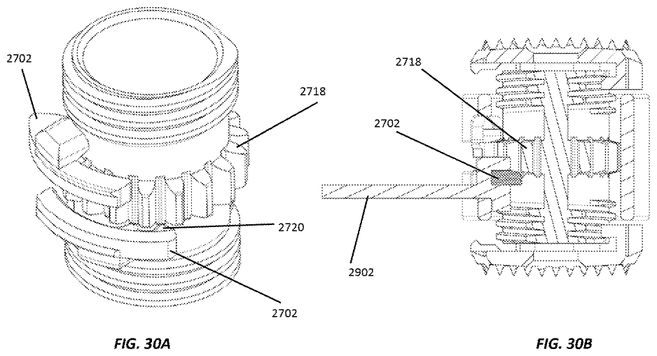

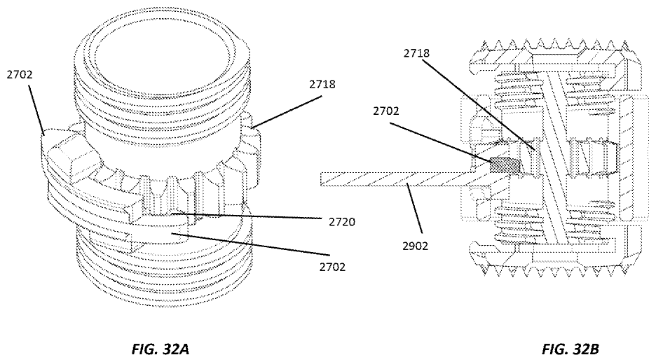

In practice, locking mechanism 2702 is positioned inside of a cavity between threaded actuator 2708, the RH End 2710, and LH End 2712 of assembly 2700. In the process of attaching an insertion instrument 2902 to outer ring 2704 (see FIG. 29), teeth of locking mechanism 2702 are disengaged from gear 2718 of the threaded actuator 2708 as the lock is moved into a neutral position around threaded actuator 2708. This is illustrated in FIGS. 30A and 30B. During the process of removal of insertion instrument 2902 from assembly 2700, teeth 2720 of locking mechanism 2702 are moved into position in gear 2718 teeth of threaded actuator 2704. This is shown in FIG. 31. Assembly 2700 is thereby prevented from contraction and expansion as shown in FIGS. 32A and 32B.

To increase torsional strength of assembly 2700, outer ring 2704 may be added to assembly 2700. Outer ring 2704 may be disposed over splines of the RH end 2710 and LH end 2712. This will assist in keeping the splines in their positions relative to one another and prevent splaying under a torsional load.

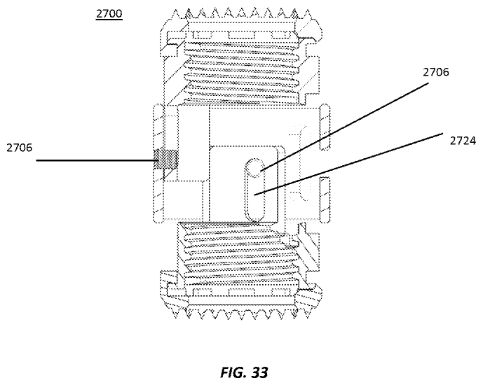

To limit the range of expansion of assembly 2700, retaining pins 2706 may be disposed in mating holes 2722 in outer ring 2704 and move within a length of mating slots 2724 in the RH and LH Ends, as shown in FIG. 33. In FIG. 33, retaining pins 2706 may be firmly assembled into outer ring 2704 and forming a positive stop to prevent disassembly of assembly 2700. Assembly 2700 is shown in a fully expanded condition with pins 2706 positioned in mating slots 2724 in RH end 2710 and LH end 2712. RH end 2710 and LH end 2712 are shown confined within an internal area of the outer ring 2704 to increase torsional strength of implant.

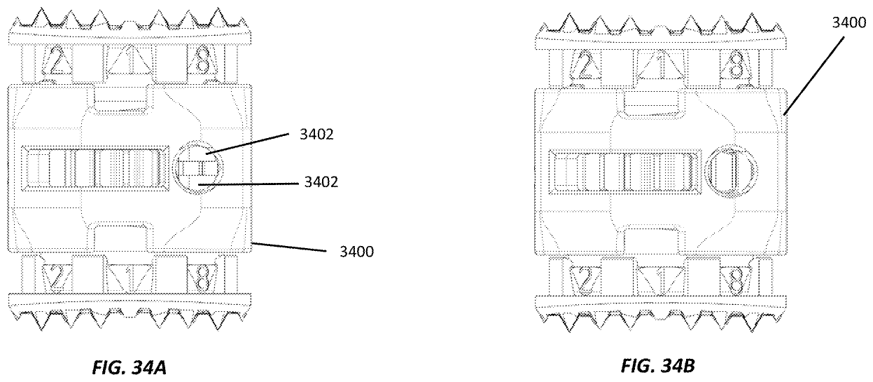

Referring now to FIGS. 34A and 34B, illustrated is an assembly 3400 consistent with the principles of the present disclosure. FIG. 34A shows assembly 3400 in a locked position and FIG. 34B shows assembly 3400 in an unlocked position. Assembly 3400 may contain the same or similar components as previously noted and contains a locking mechanism 3402 to automatically lock assembly 3400. Locking mechanism 3402 may automatically lock a threaded implant in any position to prevent it from moving, winding down or collapsing from weight, external interference, movement or vibration. Locking mechanism 3402 is seated within an interior portion of assembly 3400, with a visible gage to indicate that locking mechanism 3402 is in a locked or unlocked position. The visual indicator may be that locking mechanism 3402 is anodized to a contrasting color for visual confirmation that locking mechanism is engaged with the gear teeth. The lock functions in conjunction with the drive gear of the threaded actuator.

When the locking mechanism 3402 is engaged, locking mechanism 3402 visually seats within the gear teeth of the actuator. This engaged position prevents the actuator from rotating. In the unlocked position of FIG. 34B, locking mechanism 3402 is splayed open to vacate the space between the gear teeth. This splayed, disengaged position allows the gear of the inserter to rotate the actuator gear of the implant for expansion or contraction of the implant. Locking mechanism 3402 functions as a spring so that it can automatically re-engage the actuator gear teeth once released from this splayed position.

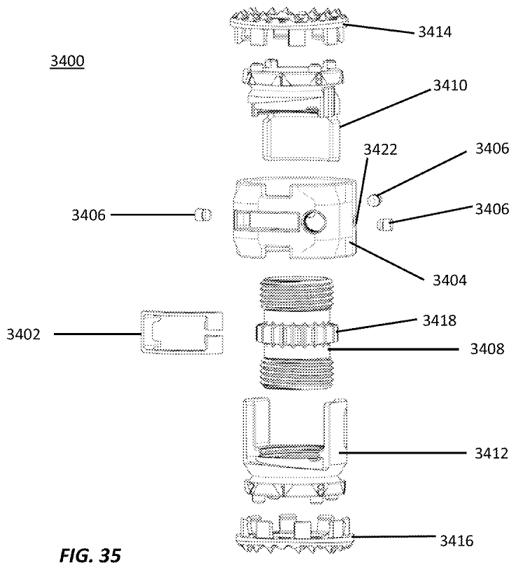

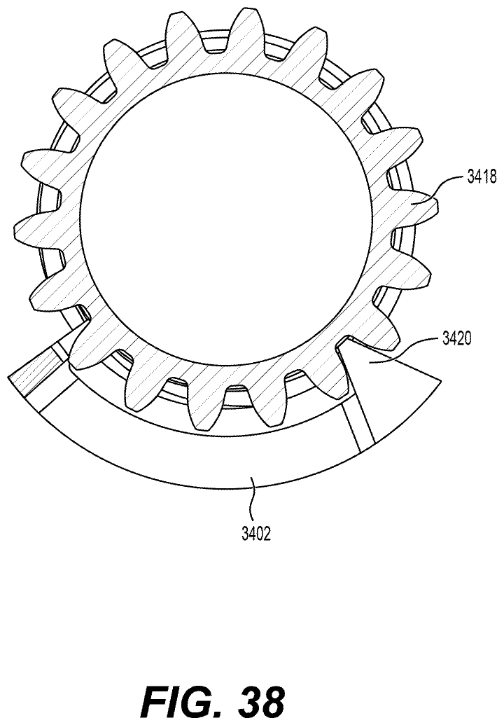

An exploded view of assembly 3400 is shown in FIG. 35. Assembly 3400 may include locking mechanism 3402, an outer ring 3404, one more retaining pins 3406, a threaded actuator 3408, a right hand (RH) end 3410, a left hand (LH) end 3412, and endplates 3414, 3416 configured to engage vertebral bodies. Threaded actuator 3408 may have gear 3418. Locking mechanism 3402 may have teeth 3420 as shown in FIG. 38. Outer ring 3404 may have a mating hole 3422 configured to receive one of retaining pins 3406.

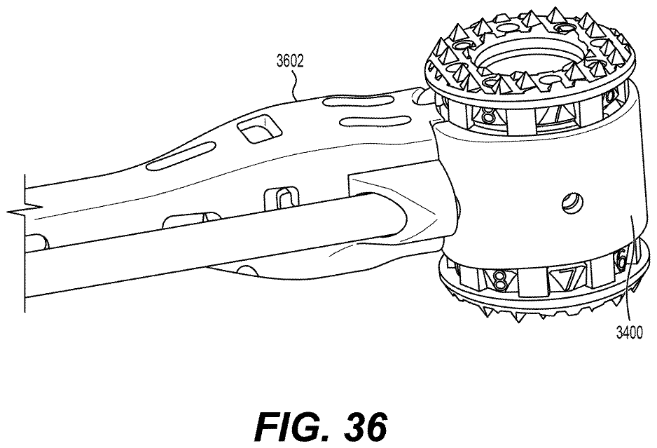

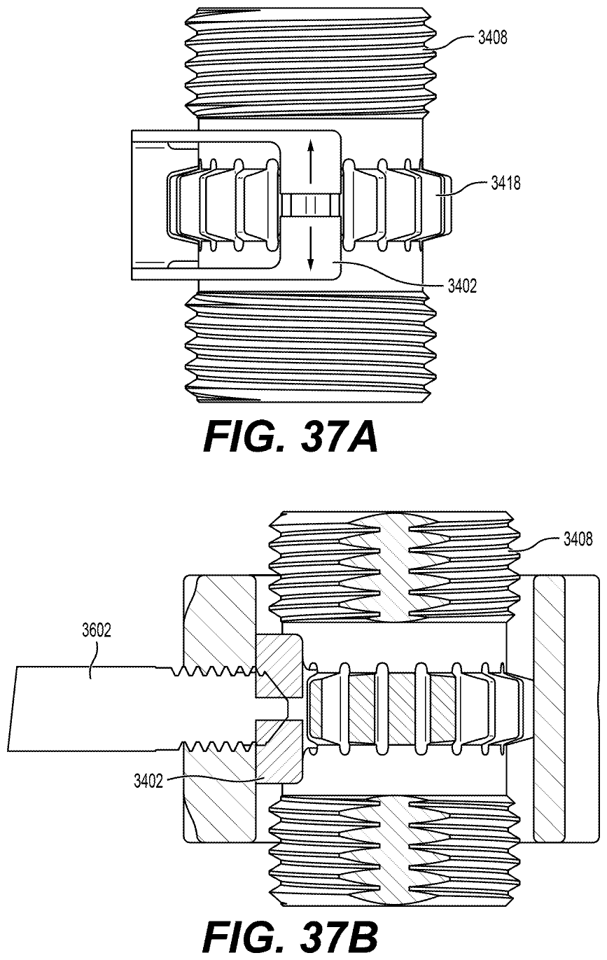

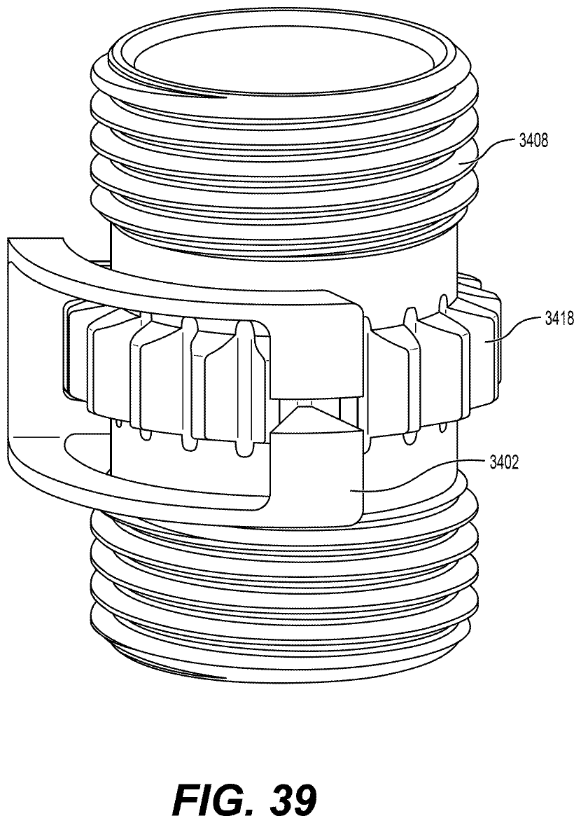

In practice, locking mechanism 3402 is positioned inside of a cavity between threaded actuator 3408, the RH End 3410, and LH End 3412 of assembly 2700. In the process of attaching an insertion instrument 3602 to outer ring 3404 (see FIG. 36), teeth of locking mechanism 3402 are splayed to disengage from gear 3418 of the threaded actuator 3408 as locking mechanism 3402 springs outward away from gear teeth 3418 of the threaded actuator 3408. This is illustrated in FIGS. 37A and 37B. Removing insertion instrument 3602 from assembly 3400 allows locking mechanism 3402 to automatically spring back into position to re-engage gear teeth 3418 of threaded actuator 3408. Locking mechanism 3402 is now engaged and assembly 3400 is prevented from contracting and expanding. See FIGS. 38 and 39.

To increase torsional strength of assembly 3400, outer ring 3404 may be added to assembly 3400. Outer ring 3404 may be disposed over splines of RH end 3410 and LH end 3412. This will assist in keeping the splines in their positions relative to one another and prevent splaying under a torsional load.

An advantage of the assembly is that locking of the assembly may occur automatically once an insertion instrument is removed. The lock is not subject to surgical approach, visual alignment or physical access to a separate locking screw that may be difficult for the surgeon to see. The lock may also form a visual confirmation by blocking a view of the drive gear of the assembly giving the surgeon visible confirmation that the implant is locked. The lock, while manufactured as one component, effectively acts as two independent locking teeth, providing additional security in the event that one lock tooth is damaged or malfunctions.

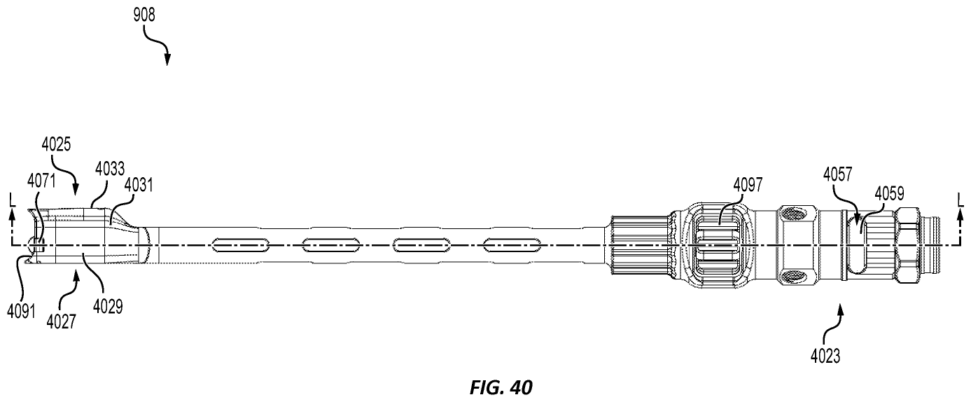

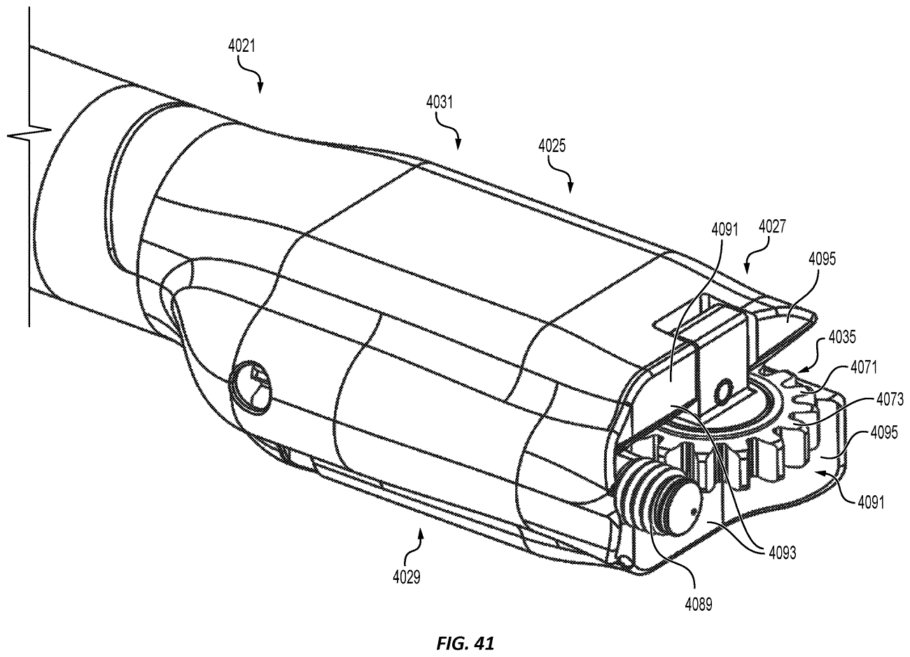

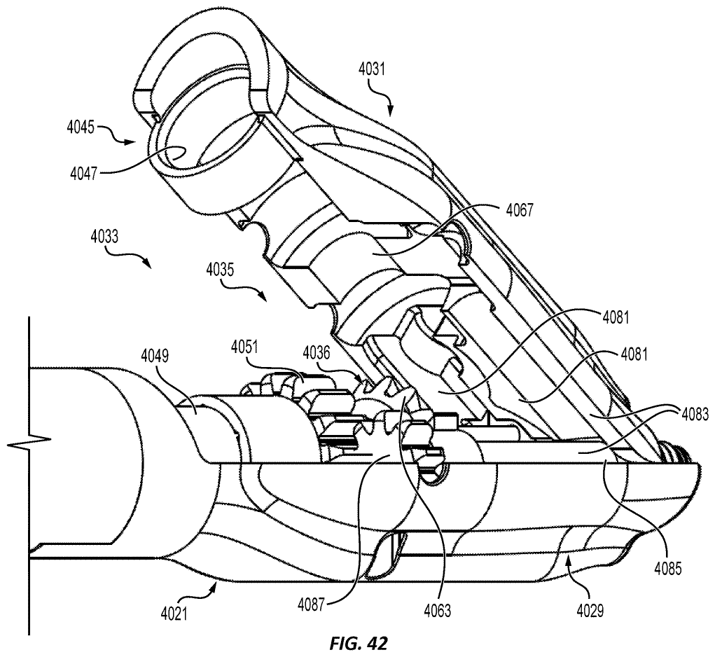

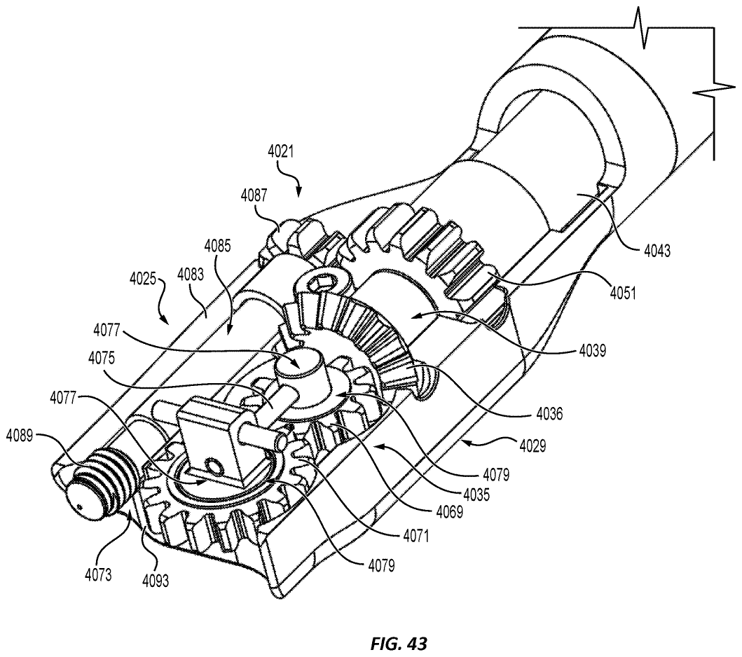

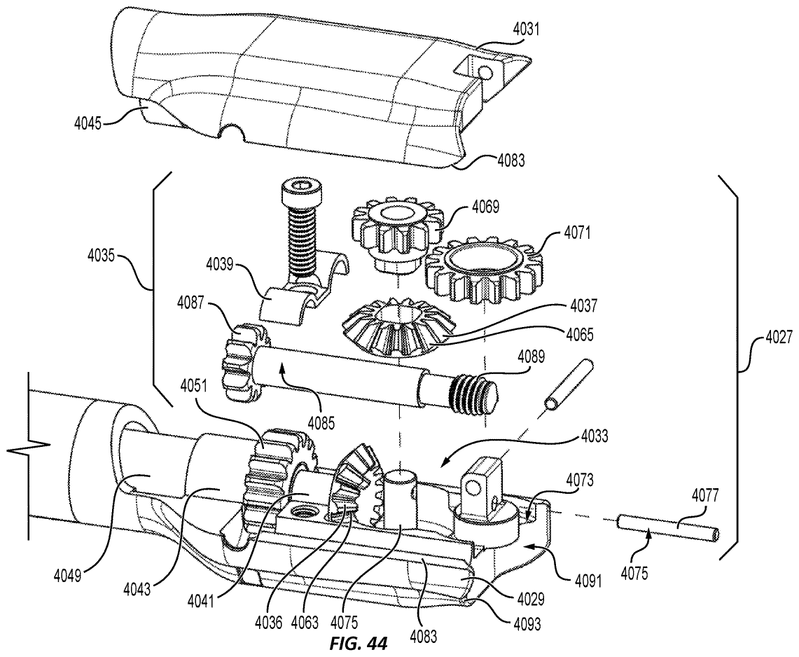

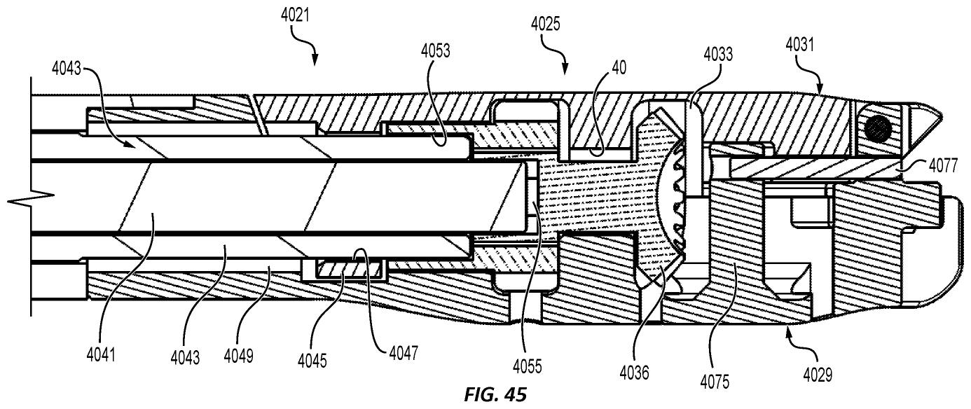

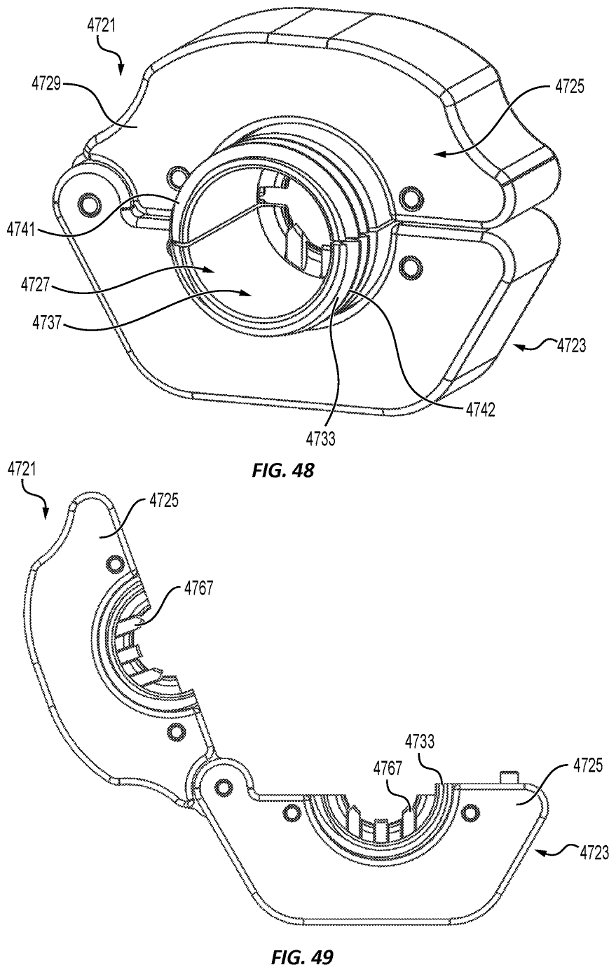

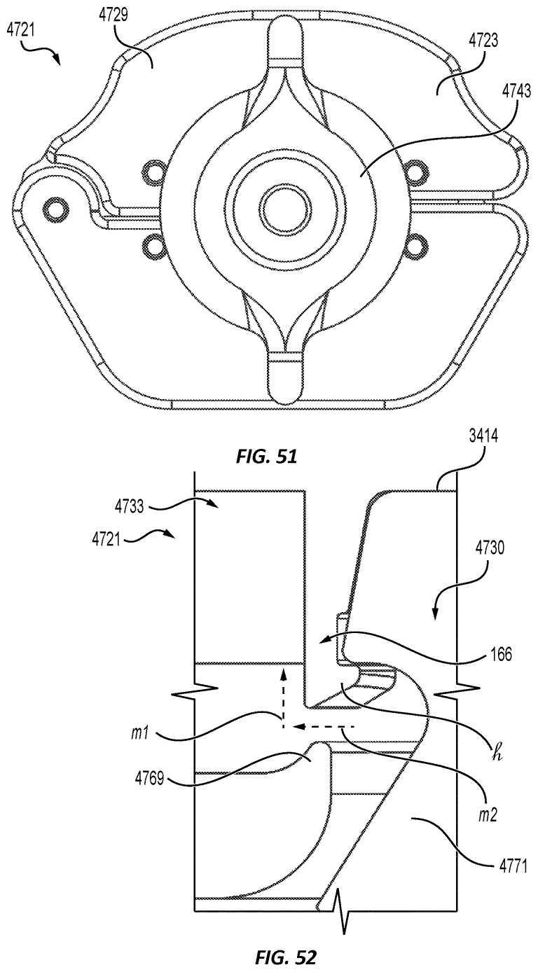

Still other possible implementations of a corpectomy system are shown and described with reference to FIGS. 40-54. Referring now to FIGS. 40-46, an insertion instrument 4021, according to one possible implementation of this disclosure, is adapted to surgically implant an implant assembly, such as implant assemble 3400 (FIGS. 34A, 34B), between two vertebral bodies, such insertion instrument generally being used as part of a corpectomy system and associated procedures. Insertion instrument 4021 has a longitudinal axis L extending between proximal and distal ends 4023, 4025. A split enclosure 4027 is located at distal end 4025 of insertion instrument 4021.

Split enclosure 4027 has a base portion 4029 and a housing cover 4031 hingedly connected relative to the base portion 4029. In this case, the hinged connection is at the forward end of distal end 4025 so that cover 4031 can be selectively opened. The base portion 4029 and cover 4031 thus form a housing 4033 which can be selectively opened and closed by a user from a suitable position relative to distal end 4025 by opening cover 4031for access or operations.

When housing 4033 is opened, a gearing mechanism 4035 disposed therein may be accessed. Gearing mechanism 4035 includes a first bevel gear 4036 and a second bevel gear 4037 operatively connected to first bevel gear 4036. First bevel gear 4036 is mounted and longitudinally oriented in relation to longitudinal axis L within housing 4033. Accordingly, as discussed subsequently, first bevel gear 4036 may be selectively rotatable about longitudinal axis L of insertion instrument 4023. Second bevel gear 4027 is mounted orthogonally to longitudinal axis L.

Insertion instrument 4021 includes a retaining member 4039 disposed within housing 4033 and secured relative to first bevel gear 4036 to constrain the longitudinal orientation of such first bevel gear 4036 against non-longitudinally oriented, bending moments which would otherwise risk disengagement, partially or fully, between first bevel gear 4036 and second bevel gear 4037.

Insertion instrument 4021 includes longitudinally extending inner and outer driving shafts 4041, 4043 which have respective distal portions receivable through a proximal aperture in the housing and selectively insertable and removable into housing 4033, by either advancing the driving shafts 4043, 4043 for insertion or retracting them longitudinally relative to housing 4033. Certain structures or components of insertion instrument 4021 allow such driving shafts 4041, 4043 to assist in locking housing 4033 from inadvertent opening. Insertion instrument 4021 likewise includes features to retain driving shafts 4041 and 4043 in an engaged position for procedures using insertion instrument 4021.

More specifically, insertion instrument 4021, as shown, may make use of a retention ring 4045 which extends inwardly from housing cover 4031 (FIG. 42). Retention ring 4045 is oriented to receive driving shafts 4041, 4043 therethrough, and has an inner surface 4047 opposing outer driving shaft 4043. In this way, contact between inner surface 4047 of retention ring 4043 and outer surface 4049 of outer driving shaft 4043 prevents housing cover 4031 from opening.

With regard to selectively attaching and detaching driving shafts 4043, 4044 to housing 4033, a proximal gear 4051 is also disposed within housing 4033 and rotatable about longitudinal axis L at a proximal location relative to first bevel gear 4035. As such, distal end of outer driving shaft 4043 is removably securable to such proximal gear 4051 by means of a first, keyed connection 4053. This first keyed connection allows rotation of outer driving shaft 4043 to be transmitted directly to rotation of such proximal gear 4053. The key connection 4053 also may be selectively disconnected so that outer driving shaft 4043 may be withdrawn proximally from engagement with proximal gear 4051 and removed completely from housing 4033.

Similarly, the distal end of inner driving shaft 4041 is removably secured to the first bevel gear 4036 discussed previously via a second keyed connection 4055. Second keyed connection 4055, similarly to first keyed connection 4053, is adapted to transmit rotation of inner driving shaft 4041 directly into rotation of first bevel gear 4036, and may likewise be selectively disconnected form first bevel gear 4036 and withdrawn proximally from housing 4033.

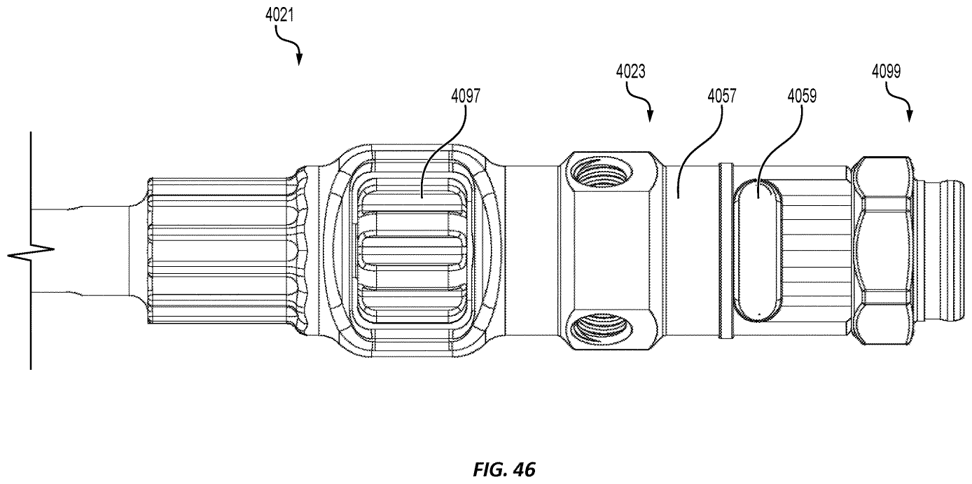

In accordance with further possible implementations of this disclosure, insertion instrument 4021 may be equipped with a sensor 4057 (FIGS. 40, 46) to detect previous use of instrument 4021 on an associated procedure. Sensor 4057 may have suitable mechanical or electronic triggers to indicate, in particular, previous actuation of gearing mechanism 4035, along with a suitable indicator 4059, such as a light or other visual indication. In the illustrated implementation, both sensor 4057 and indicator 4059 are disposed to be visible to a user at the proximal end 4023 of insertion instrument 4021, though of course other locations and sensing arrangements are likewise suitable. In this way, in response to the indication of previous use, the instrument may be cleaned generally, or, more particularly, the gearing mechanism may be cleaned.

One suitable cleaning procedure available would involve disconnecting inner and outer driving shafts 4041, 4043 from associated keyed connections 4053 and 4055, as discussed previously, withdrawing the driving shafts proximally from housing 4033. By withdrawing driving shafts 4041, 4043 in this manner, the engaging contact between retention ring 4045 and outer driving shaft 4043, described previously with respect to FIG. 42, is eliminated, which thereafter permits housing 4033 to be opened, whereupon gearing mechanism 4035 may be accessed and cleaned.

By virtue of its location proximally within housing 4033, first bevel gear 4036 may take the form of an initial, bevel gear 4063, and second bevel gear 4037 may take the form of receiving bevel gear 4065. Retaining member 4039 may assume any number of forms so long as it operatively retains inner driving shaft 4041 in proximity to initial bevel gear 4063. Retaining member 4039 may comprise a boss 4067 formed on an inner surface of housing cover 4031 (FIG. 42). In such implementation, boss 4067 is disposed at a location in operative proximity to initial bevel gear 4063, as mentioned previously, and is configured to exert opposing force against non-longitudinal bending moments on the inner driving shaft 4043.

Housing 4033 includes still further features disposed therein to enhance the robustness of gearing mechanism 4035 during contemplated corpectomy operations. For example, a reinforcing element 4075 is disposed within housing 4033 and extends longitudinally between the longitudinally spaced locations of a first spur gear 4069 and a second spur gear 4071. Connection of reinforcing element 4075 relative to first and second spur gears 4069, 4071 constrains non-longitudinal movement of first spur gear 4069 relative to second spur gear 4071 when distal end 4025 of insertion instrument 4021 experiences non longitudinal bending moments. Reinforcing element 4075 is in the form of a pin extending longitudinally between the respective, rotational axes of first and second spur gears 4069, 4071. The pin is removably insertable through bores extending through the mounting locations of such first and second spur gears.

Spur gears 4069, 4071 have transversely oriented flanks 4079 and teeth extending between such flanks. In certain implementations, housing cover 4031 may be configured to opposed such flanks 4079 but not inhibit normal rotation of spur gears 4069, 4071 under the range of anticipated operations of insertion instrument 4021. So, in the illustrated implementation, housing cover 4031 may have shoulders 4081 with surfaces in slightly spaced but opposing relationship to corresponding flanks 4079 of second spur gear 4071. Such shoulders 4081, while not normally interfering with rotation of second spur gear 4071, will be useful in instances when insertion instrument 4021 is subjected to certain bending moments or other forces during use, which would otherwise cause second spur gear 4071 to misalign or undesirably move relative to first spur gear 4069. The foregoing structures thus permit operations of insertion instrument 4021 while resisting undesirable bending moments when experienced during such operations.

The enclosing aspects of housing 4033 are accomplished by configuring hosing cover 4031 and base portion 4029 to have opposing mating surfaces 4083. Opposing mating surfaces 4083 are disposed at respective locations on housing cover 4031 and base portion 4029, such mating surfaces 4083 being separable only upon opening of housing cover 4031. Retention mechanism 4044, in the form of retention ring 4045 discussed previously, likewise serves to maintain the enclosed state of housing 4033 by holding mating surfaces 4083 in opposing relationship to prevent or at least reduce entry of undesired foreign material into housing.

In accordance with certain implementations of the present disclosure, insertion tool 4021 may have an actuation shaft 4085 extending longitudinally within housing 4033 and rotatably connected to outer driving shaft 4043. As such, actuation shaft 4085 can be rotated in response to selective rotation of outer driving shaft 4043. Actuation shaft 4085 has a distal shaft end extending distally through an engagement aperture 4073 at distal end 4025 of insertion instrument 4021. Similarly, second spur gear 4071 has an arcuate portion 4073 extending distally through engagement aperture 4073.

The distal shaft end of actuation shaft 4085 and distal arcuate portion 4073 of second spur gear 4071 may be brought against an implant assembly through engagement aperture 7073, such as implant assembly 3400 (FIGS. 34A, 34B) or other assemblies disclosed herein, and thereby be selectively engaged at corresponding locations on implant assembly 3400. The distal shaft end of actuation shaft 4085 has a threaded connection 4089 formed thereon to operatively engage a corresponding location on a suitable implant assembly, such as implant assembly 3400. Rotation of threaded connection 4089 is accomplished by transmitting rotation of outer driving shaft 4043 through a proximal gear 4041, which proximal gear 4041 is meshed with an actuation shaft gear 4087 at the proximal end of actuation shaft 4085.

Distal end 4025 of insertion instrument 4021 has an engagement surface 4091 thereon, such engagement surface 4091 defining the above-mentioned engagement aperture 4073. Engagement surface 4091 is contoured to engage corresponding implant assemblies which may be selected from any number of different diameters for such implant assemblies, such as implant assembly 3400 (FIGS. 34A, 34B). In the illustrated implementation, engagement surface 4091 has a seat 4093 positioned in operative proximity to the distal end of actuation shaft 4085 and configured to receive the implant assembly thereon. As disclosed herein with reference to FIGS. 34-39, locking mechanisms 3402, outer ring 3404 (FIG. 35) (FIG. 35) and other related or operable features of implant assemblies may be brought into operative proximity to seat 4091 for suitable retention, engagement and operations, including having a locking mechanism being selectively splayed open by rotation of actuator shaft 4085.

Engagement surface 4091 is formed to have a cradle portion 4095 disposed adjacent to seat 4093. As such, cradle portion 4095 may have a curved surface selected to oppose the outer ring 3404 (FIG. 35) such curved surface contoured so that implant assemblies having different diameters may be supported thereby.

Inner and outer driving shafts 4041, 4043 have respective proximal portions extending to the proximal end 4023 of insertion instrument 4021. A manually actuatable thumb wheel 4097 is connected to outer driving shaft 4043 and is manually accessible at proximal end 4023. Manual actuation of thumb wheel 4097 in a rotating manner (or a geared or electronic equivalent thereto (not shown)) transmits rotation to outer driving shaft 4043, which in turn transmits rotation to actuation shaft 4085, which in turn transmits rotation to a corresponding locking mechanism associated with an implant assembly, such as locking mechanism 3402 of implant assembly 3400 (FIGS. 34A, 34B). Rotation of actuation shaft 4085 is thus used to open portions of locking mechanism 3402.

Also at proximal end 4023 of insertion instrument 4021, a connector 4099 makes inner driving shaft 4041 accessible to a suitable rotation or drive mechanism (not shown). Through connector 4099, inner driving shaft 4041 may be rotated from its proximal end, which rotation is transmitted to housing 4033, causing selective rotation of second spur gear 4071 and its protruding arcuate portion 4073. Inner driving shaft 3031 thus may be selectively actuated to perform suitable operations on implant assembly 3400 by virtue of rotation of second spur gear 4071. Thus, for example, inner driving shaft 3031 may be used for expansion, contraction, or other operations relating to the components of implant assembly 3400, including the relative rotations of components in relation to threaded actuator 3408 (FIG. 38).

Operations of insertion instrument 4021 are readily apparent from the foregoing description. In general terms, thumb wheel 4097 may be selectively rotated by the medical practitioner or other user to drive outer driving shaft 4043 to attach or detach a suitable implant assembly to insertion instrument 4021 and may be further actuated to splay open or otherwise operate a locking mechanism associated with the implant assembly, such as locking mechanism 3402. Inner driving shaft 4041 may be suitable operated, such as by rotation, which operations are transmitted to housing 4033 and its distally located second spur gear 4071 to expand or contract an implant assembly operatively engaged by insertion instrument 4021 in performance of related corpectomy procedures.

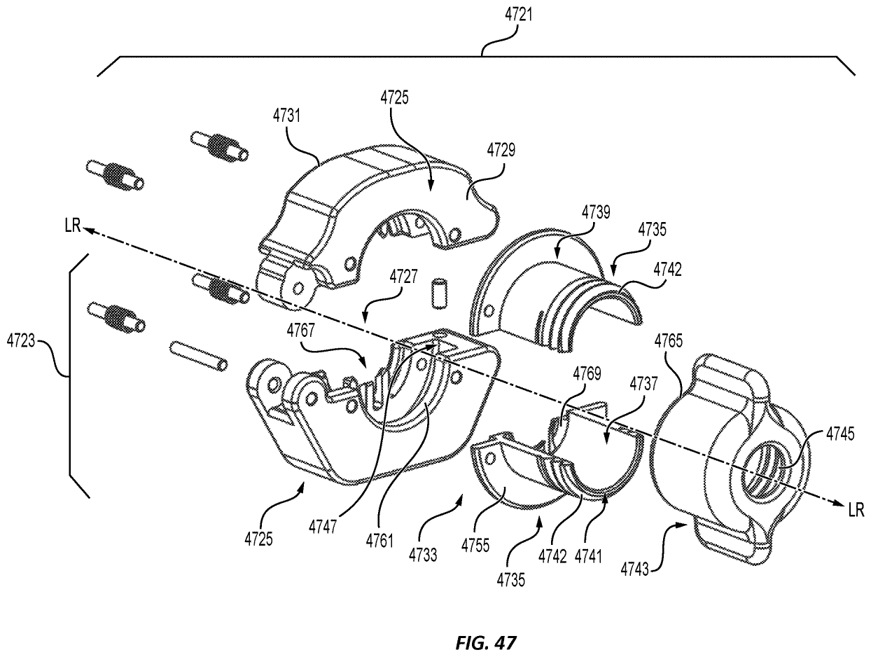

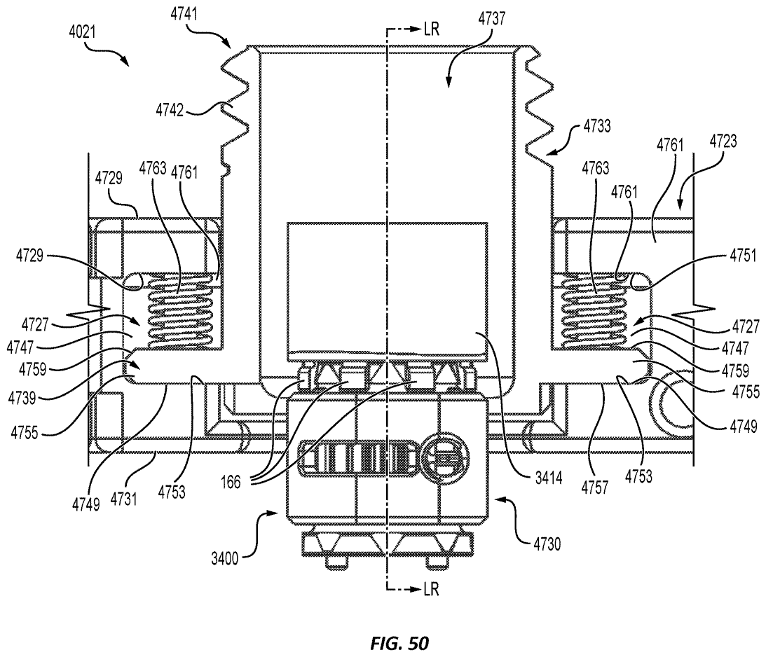

Referring now more particularly to FIGS. 47-53, in certain implementations of the present disclosure, a corpectomy system may include a suitable removal tool 4721 for use in non destructively separating an end cap from a core of an implant assembly. By way of example, and not in any way limiting the generality and usefulness of removal tool 4721, suitable implant assemblies for use with removal tool 4721 may have end caps such as those disclosed herein as end caps 3414, 3416 (FIG. 35), while cores 4730 (FIG. 50) may assume any of variety of forms, such as that formed by threaded actuator 3408, outer ring 3404, and right-hand and left-hand ends 3410, 3412 (FIG. 35). Suitable end caps will have resiliently flexible tabs, such as flexible tabs 166 (FIGS. 6-9, FIG. 50), which are operated upon as explained herein to nondestructively separate the end cap from its attachment location on core 4730.

One suitable structure and configuration for accomplishing the foregoing objectives, according to the present disclosure, may include a split clamp 4723 having first and second, C-shaped clamp portions 4725 with hingedly connected opposing ends. Such clamp portions 4725 have respective concave surfaces, and the clamp portions are rotatable relative to each other to open and close the split clamp 4723. The concave surfaces form a clamp aperture 4727 having an inner surface extending circumferentially around a longitudinal axis LR when closed, and extending longitudinally between first and second transverse clamp sides 4729, 4731.

Removal tool 4721 includes a split ring 4733 connected to split clamp 4723. Split ring 4733 is formed to have first and second split ring portions 4735 which correspond to the first and second clamp portions 4725. Split ring portions 4735 are connected so that when split clamp 4723 is opened, the split ring portions are likewise opened. Accordingly, split ring 4733 defines an inner split ring aperture 4737 when closed.

To accomplish its operations, split ring aperture 4737 and clamp aperture 4727 are configured and sized to operatively engage a suitable core, such as core 4730 illustrated herein, including engagement or operative proximity (as explained herein) to tabs 66 of the end cap 3414. When split clamp 4723 and split ring4733 are closed around the implant assembly 3400 (FIGS. 34A, 34B) at a predetermined longitudinal location on such implant assembly 3400. Split ring 4733 has two opposite ends, the first end 4739 is connected to the split clamp 4723 in a manner that permits split ring 4733 to be longitudinally displaceable relative to split clamp 4723. Second end 4741 of split ring 4733 has a threaded portion 4742 extending longitudinally between the first and second ends of split ring 4733.

A removal cap 4743 has threads thereon which correspond to those of threaded portion 4742 of split clamp 4733 and is thereby advanceable on threaded portion 4742 toward one of the sides of split clamp 4723, in the illustrated implementation, toward first transverse clamp side 4729. In response to sufficient advancement of removal cap 4743 toward and against transverse clamp side 4729, the first end 4739 of split clamp 4723 is translated relative to the split clamp 4723 so as to non destructively disengage tabs 166 from core 4730.

More specifically with reference to FIG. 50, split ring 4733 includes a flange 4755 which is received within a circumferential channel 4747 and longitudinally displaceable within such channel. Spring means 4763, in this case multiple, circumferentially spaced helical springs, are operatively received in circumferential channel 4747 to bias flange 4755 forward relative to the clamp aperture 4727. Removal cap 4743, when sufficiently rotated, bears against the surface of transverse clamp side 4729, pulls flange 4755 against the spring bias, with portions of split ring 4733 disengaging tabs 166, and with portions of clamp aperture 4727 holding core 4730 during such separating operation, so that the end cap may be non-destructively removed therefrom.

Further describing the illustrated structures for accomplishing the foregoing, again, by way of example, and in no way limiting variations and alternative implementations, channel 4747 may be formed with first and second opposing channel walls 4749, 4751 which extends circumferentially relative to longitudinal axis LR. For its part, flange 4755 has forward and rearward facing surfaces 4755, 4759 which oppose first and second opposing channel walls 4749, 4751, as best seen in FIG. 50. Channel wall 4749 comprises a seat 4753 which against flange 4755 is biased by spring means 4763 which is disposed between rearward facing surface 4759 of flange 4755 and second channel wall 4751. As such, second channel wall 4751 comprises a spring wall 4761 with a first side of the spring wall facing channel 4747 and having spring means 4763 engage therewith, and with the second outer side of spring wall 4761 facing an opposing surface of removal cap 4753 when removal cap is rotatably engaged on spilt ring 4733. In view of the foregoing, the helical springs of spring means 4763 extend between spring wall 4763 and rearward surface 4759 of flange 4755 to bias forward surface 4757 against seat 4753.

Removal cap 4753 has a bearing surface 4765 oriented to face the side of spring wall 4761 which opposes it. The threaded portion 4742 of removal cap 4743 is disposed and sufficiently long so that, upon advancement of threaded removal cap 4745, bearing surface 4765 first advances to engage spring wall 4761 and, upon further advancement, the rearward surface of flange 4755 exerts force against the spring bias to displace flange 4755 toward spring wall 4761 and proximally relative to the core 4730 received in the removal tool 4721.

When flange 4755 is thus pulled or urged upwardly (when in the orientation shown in FIG. 50), a set of teeth 4769 which extend inwardly from split ring aperture 4737 are moved in the direction m1, as shown in FIG. 52, to operatively engage opposing ones of tabs 166. during advancement of removal cap 4743. Thereafter, upon further advancement, certain portions 4771 of core 4730 are brought into engagement with teeth 4769 to cooperate to urge teeth 4769 against opposing head surfaces h of tabs 166. The configuration of head surfaces h are configured, in this case sloped upwardly and proximally, to cause the tabs 166 to move in the direction m2 and thereby cause their nondestructive disengagement from core 4730.

During the foregoing operations, another set of teeth 4767, which extend radially inwardly from clamp aperture 4727, are sufficiently engaged with core 4730 so that, during the pulling away or longitudinal displacement of the set of teeth 4769 on split ring aperture 4737, the core 4730 is held in position so that the force in direction m2 on end cap 3414 and its tabs 166 is effective to separate tabs 166 from core 4730.

Various operations of removal tool 4721 are apparent from the foregoing description. An implant assembly, such as one with a core 4730 and an end cap 3414 thereon, is received by opening split clamp 4723 and spilt ring 4733, positioning the implant assembly at a suitable longitudinal location therein. One such suitable location has the teeth on split ring 4733 in operative proximity or engagement with tabs 166 of end cap 3414 and the teeth on clamp aperture 4727 engage opposing portions of core 4730 when the split clamp and split ring are closed around the implant assembly. Thereafter, removal cap 4745 is threadably received on threads 4742 of the split ring 4733 and advanced by turning toward the opposing transverse side 4729 of split clamp 4723.

A bearing surface on removal cap 4743 ultimately engages the opposing transverse side 4729 of split clamp 4723. Further advancement against the transverse side 4729 overcomes the bias of the spring or springs received in the channel, unseating flange 4755. The teeth 4769 engage opposing portions of heads h on tabs 166 and pull tabs 166 out of engagement with a bezel or other retaining structures on the opposing surface of core 4730. Once end cap 166 has been separated as described above, split clamp 4723 and spring ring 4733 can be opened to disengage core 4730 and remove core and end cap 3414 from the removal tool, whereupon end cap 3414 may be reused in another application.

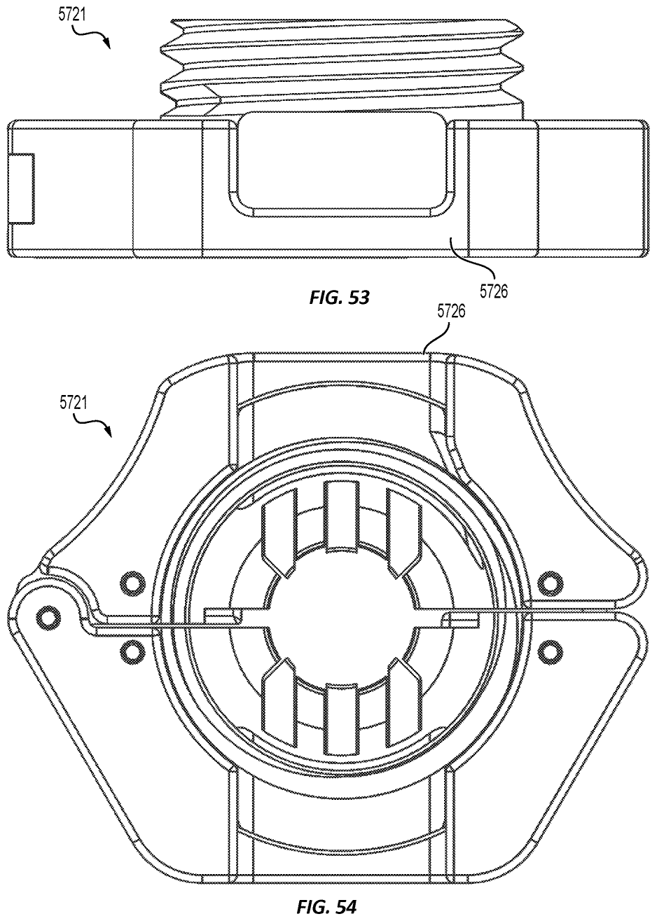

The absolute and relative dimensions of removal tool 4721 may be varied to suit any number of applications, including different sizes and configurations of implant assemblies and endplate configurations. As one further, non-limiting example, referring to FIGS. 53 and 54, a removal tool 5721 may have larger apertures formed thereon for correspondingly larger implants, such as by elongating the apertures, or separating the C-shaped portions from each other, by means of a spacer section element 5726. Still further variations to the removal tool may be made for use with lateral end-plates and corresponding cores.

These and other advantages of the present disclosure will be apparent to those skilled in the art from the foregoing specification. Accordingly, it will be recognized by those skilled in the art that changes or modifications may be made to the above-described embodiments without departing from the broad inventive concepts. It should therefore be understood that this disclosure is not limited to the particular embodiments described herein, but is intended to include all changes and modifications that are within the scope and spirit of the disclosure as defined in the claims.

What is claimed is:

1. A corpectomy system, the system comprising:
an implant assembly for engagement between a first vertebral body and a second vertebral body, the implant assembly having first and second opposite ends and at least one endplate configured to be removably attachable to one of the ends of the implant assembly;
an insertion instrument adapted to surgically implant the implant assembly, the insertion instrument extending between proximal and distal ends and having a longitudinal axis defined therebetween;
wherein the insertion instrument includes a split enclosure at the distal end, the split enclosure comprising a base portion and a housing cover hingedly connected relative to the base portion, the base portion and the housing cover defining a housing, the housing having a gearing mechanism disposed therein and accessible when the housing cover is open;
wherein the implant assembly includes:
an outer ring;
a threaded actuator disposed inside the outer ring and having an actuator gear;
a right-hand end configured to engage the threaded actuator;
a left-hand end configured to engage the threaded actuator; and
a locking mechanism disposed in the outer ring and configured to removably engage with the actuator gear, wherein the locking mechanism is a single component that is configured to engage with the actuator gear in a locked position and is configured to splay open and disengage from the actuator gear in an unlocked position; and
wherein the at least one endplate comprises a plurality of tabs positionable to oppose a corresponding one of the ends of the implant assembly, the tabs disposed at spaced locations about the circumference of the endplate to be releasably engageable at corresponding, opposing locations on the end of the implant assembly.

2. The system of claim 1,
wherein the gearing mechanism comprises a first bevel gear and a second bevel gear operatively connected to the first bevel gear, the first bevel gear mounted and longitudinally oriented within the housing to be selectively rotatable about the longitudinal axis of the insertion instrument, the second bevel gear mounted orthogonally to the longitudinal axis;
wherein the housing includes a retaining member secured relative to the first bevel gear to constrain the longitudinal orientation of the first bevel gear against non-longitudinally oriented bending moments to minimize risk of reduced operative engagement between the first bevel gear and the second bevel gear.

3. The system of claim 2,
wherein the insertion instrument comprises longitudinally extending inner and outer driving shafts with respective, distal portions removably insertable into the housing;
wherein the first bevel gear comprises an initial bevel gear connected to the inner driving shaft and the second bevel gear comprises a receiving bevel gear distally mounted relative to the initial bevel gear.

4. The system of claim 3, wherein the retaining member comprises a retaining clip opposing the inner driving shaft and disposed in operative proximity to the initial bevel gear.

5. The system of claim 3, wherein the retaining member comprises a boss formed on an inner surface of the housing cover, the boss disposed at a location in operative proximity to the initial bevel gear to exert opposing force against non-longitudinal bending moments on the inner drive shaft.

6. The system of claim 2,
wherein the gearing mechanism includes first and second spur gears mounted at respective, longitudinally spaced locations at the distal end;
wherein the insertion instrument comprises an engagement aperture at the distal end, the second spur gear having a distal, arcuate portion extending distally through the engagement aperture to selectively engage the implant assembly; and
wherein the insertion instrument comprises a reinforcing element extending between the longitudinally spaced locations of the first and second spur gears, the reinforcing element being configured to constrain non-longitudinal movement of the first spur gear relative to the second spur gear when the distal end of the insertion instrument experiences non-longitudinal bending moments.

7. The system of claim 6, wherein the spur gears have respective mounts disposed at spaced locations within the housing, the reinforcing element comprising a pin extending longitudinally between the respective, rotational axes of the first and second spur gears, the pin removably insertable through bores extending through the mounts.

8. The system of claim 7,
wherein the spur gears have transversely oriented flanks and teeth extending between the flanks;
wherein the housing cover is hingedly connected at a distal location corresponding to the mount of the second spur gear, the housing cover having shoulders with surfaces opposing a corresponding one of the flanks of the second spur gear to constrain the second spur gear from disengaging movement relative to the first spur gear.

9. The system of claim 1,
wherein the insertion instrument comprises longitudinally extending inner and outer driving shafts with respective distal portions receivably insertable into the housing;
wherein the housing cover and the base include mating surfaces disposed at a longitudinally spaced location relative to the hinged connection of the housing cover, the mating surfaces separable upon opening of the housing cover, the mating surfaces in respective, opposing relationships to each other when the housing cover is closed; and
wherein the instrument comprises a retention mechanism to hold the mating surfaces in the opposing relationship to prevent the housing cover from opening.

10. The system of claim 9,
wherein the retention mechanism comprises a retention ring extending inwardly from the housing cover, the ring being oriented to receive the driving shafts therethrough, the retaining ring having an inner surface opposing the outer driving shaft, whereby contact between the inner surface of the retention ring and the outer surface of the outer driving shaft prevents the cover from opening.

11. The system of claim 10,
wherein the gearing mechanism includes a proximal gear disposed within the housing and rotatable about the longitudinal axis, the distal end of the outer driving shaft being removably secured to the proximal gear via a first, keyed connection, the keyed connection adapted to transmit rotation of the outer driving shaft to cause rotation of the proximal gear, the first, keyed connection adapted to permit selective disconnection of the outer driving shaft from the proximal gear; and
wherein the distal end of the inner driving shaft is removably secured to the first bevel gear via a second, keyed connection, the second keyed connection adapted to transmit rotation of the inner driving shaft to cause rotation of the first spur gear, the second keyed connection adapted to permit disconnection of the inner driving shaft from the first spur gear.

12. The system of claim 6, further comprising an actuation shaft extending longitudinally in the housing and rotatably connected to the outer driving shaft to be rotated in respond to selective rotation of the driving shaft;
wherein the actuation shaft has a distal shaft end extending distally through the engagement aperture;
wherein the distal shaft end is configured to operatively engage the implant assembly.

13. The system of claim 12,
wherein the gearing mechanism includes a proximal gear disposed within the housing and rotatable about the longitudinal axis;
wherein the actuation shaft has an actuation shaft gear at the proximal end engaged with the proximal gear;
and wherein the distal end of the actuation shaft has a threaded connection to operatively engage the implant assembly.

14. The system of claim 12, further comprising an engagement surface in which the engagement aperture at the distal end of the housing is defined, the arcuate portion of the second spur gear extending distally from the engagement surface, the engagement surface adapted to engage predetermined ones of the implant assembly having respective, multiple, different diameters, the engagement surface extending between the sides of the housing;
wherein the engagement surface has a seat positioned in operative proximity to the distal end of the actuation shaft and configured to receive on the seat a portion of the outer ring through which the locking mechanism may be selectively splayed open by rotation of the actuator shaft; and
wherein the engagement surface comprises a cradle portion disposed adjacent to the seat, the cradle portion comprising a curved surface to oppose the outer ring of any one of the predetermined different sizes of the implant assembly.

15. The system of claim 9, wherein the inner and outer driving shafts have respective proximal portions extending to the proximal end of the insertion instrument, and wherein the insertion instrument includes a manually actuatable thumb wheel to rotate the outer driving shaft and a connector to the inner driving shaft to transmit rotation thereto.

16. The system of claim 15, wherein the insertion instrument comprises a sensor to sense previous use of the gearing mechanism, and wherein the insertion instrument comprises an indicator disposed at the proximal end to indicate the previous use.

17. An insertion instrument adapted to surgically implant an implant assembly between two vertebral bodies, the insertion instrument comprising:
proximal and distal ends and a longitudinal axis defined therebetween; and
a split enclosure at the distal end, the split enclosure comprising a base portion and a housing cover hingedly connected relative to the base portion, the base portion and the housing cover defining a housing, the housing having a gearing mechanism disposed therein and accessible when the housing cover is open;
wherein the gearing mechanism comprises a first bevel gear and a second bevel gear operatively connected to the first bevel gear, the first bevel gear mounted and longitudinally oriented within the housing to be selectively rotatable about the longitudinal axis of the insertion instrument, the second bevel gear mounted orthogonally to the longitudinal axis;
wherein the insertion instrument comprises a retaining member secured relative to the first bevel gear to constrain the longitudinal orientation of the first bevel gear against non-longitudinally oriented bending moments to minimize risk of reduced operative engagement between the first bevel gear and the second bevel gear;
wherein the insertion instrument comprises longitudinally extending inner and outer driving shafts with respective distal portions receivably insertable into the housing;
wherein the insertion instrument comprises a retention ring extending inwardly from the housing cover, the ring being oriented to receive the driving shafts therethrough, the retaining ring having an inner surface opposing the outer driving shaft, whereby contact between the inner surface of the retention ring and the outer surface of the outer driving shaft prevents the cover from opening;
wherein the gearing mechanism includes a proximal gear disposed within the housing and rotatable about the longitudinal axis, the distal end of the outer driving shaft being removably secured to the proximal gear via a first keyed connection, the first keyed connection adapted to transmit rotation of the outer driving shaft to rotation of the proximal gear, the keyed connection adapted to selectively disconnect the outer driving shaft from the proximal gear;
wherein the distal end of the inner driving shaft is removably secured to the first bevel gear via a second, keyed connection, the second keyed connection adapted to transmit rotation of the inner driving shaft to rotation of the first bevel gear and to selectively disconnect the inner driving shaft from the first spur gear;
wherein the insertion instrument comprises a sensor to sense previous use of the gearing mechanism, and wherein the insertion instrument comprises an indicator disposed at the proximal end to indicate the previous use; and
wherein, in response to indication of the previous use, the first and second keyed connections are operable to disconnect the outer and inner driving shafts, respectively;
wherein the housing has a proximal opening sized to permit withdrawal of the inner and outer driving shafts proximally therefrom to eliminate the contact between the retention ring and the outer driving shaft preventing the housing from being opened, whereupon the housing cover may be opened for cleaning of the gearing mechanism.

18. A removal tool for use with a corpectomy system, in which the corpectomy system includes an implant assembly for engagement between a first vertebral body and a second vertebral body, and in which the implant assembly has a core and at least one end cap selectively attachable to the core with resiliently flexible tabs, the removal tool comprising:
a split clamp having first and second, C-shaped clamp portions with hingedly connected opposing ends, the C-shaped clamp portions having respective concave surfaces rotatable relative to each other to open and close the split clamp, the concave surfaces forming an inner surface extending circumferentially about a longitudinal axis of the split clamp and defining a clamp aperture through the split clamp when closed, the inner surface extending longitudinally between opposite, first and second transverse clamp sides; and
a split ring having first and second split ring portions corresponding to the first and second clamp portions, the split ring portions defining an inner split ring aperture, the split ring aperture and the clamp aperture configured to operatively engage the core and the tabs of the end cap when the split clamp and split ring are closed around the implant assembly at a predetermined longitudinal location;
wherein the split ring has two opposite ends, the first end connected to the split clamp to permit the split ring to be longitudinally displaceable relative to the split clamp, the second end having a threaded portion extending longitudinally between the first and second ends;
wherein the removal tool includes a removal cap having threads thereon corresponding to the threaded portion at the second end of the split clamp and thereby advanceable on the threaded portion toward the first, transverse clamp side; and
wherein the first end of the split ring and the split clamp are operatively interconnected to non-destructively disengage the tabs from the core in response to sufficient advancement of the removal cap relative to the split ring.

19. The removal tool of claim 18,
wherein the inner surface of the aperture has a circumferential channel defined therein, the channel having first and second opposing, channel walls extending circumferentially relative to the longitudinal axis;
wherein the split ring includes a flange received within the circumferential channel and longitudinally displaceable within the channel, the flange having opposite forward and rearward facing surfaces;
wherein the first channel wall comprises a seat and the second channel wall comprises a spring wall with a first side of the spring wall facing the channel and a second side of the spring wall facing the removal cap;
wherein the removal tool further comprises spring means operatively connected between the spring wall and the rearward surface of the flange to bias the forward surface of the flange against the seat; and
wherein the threaded removal cap has a bearing surface oriented to face the second side of the spring wall, wherein the threaded portion of the removal cap is disposed and sufficiently long so that, upon advancement of the threaded removal cap, the bearing surface thereof is first advanceable to engage the second side of the spring wall opposing the bearing surface and, upon further advancement after such engagement, the rearward surface of the flange exerts force against the spring bias to displace the flange toward the spring wall and proximally relative to the core.

20. The removal tool of claim 19, in which the tabs of the end caps have heads with outwardly facing head surfaces, the removal tool further comprising a first set of teeth extending radially inwardly from the clamp aperture configured to engage opposing locations on the core and a second set of teeth extending radially inwardly from the ring aperture, the second set of teeth configured to operatively engage opposing ones of the tabs upon sufficient advancement of the removal cap against the split clamp, the first set of teeth sufficiently engaging the core to permit the second set of teeth, upon sufficient advancement, to exert proximal force upon the outwardly facing head surfaces of the tabs to cause the non-destructive disengagement of the tabs from the core.
